(19) United States
(12) Patent Application Publication
(10) Pub. No.: US 2004/0186166 A1
(43) Pub. Date: Sep. 23, 2004

(54) Cannabinoid analogs as peroxisome proliferator activated nuclear receptor gamma activators

(76) Inventors: Sumner H. Burstein, Framingham, MA (US); J. Don Chen, New Brunswick, NJ (US); Robert B. Zurier, Princeton, MA (US)

Correspondence Address: FISH & RICHARDSON PC, 225 FRANKLIN ST, BOSTON, MA, 02110, US

(21) Appl. No.: 10/741,219

(22) Filed: Dec. 19, 2003

Related U.S. Application Data
(60) Provisional application No. 60/435,184, filed on Dec. 19, 2002.

(51) Int. Cl. A61K 31/353

(52) U.S. Cl. 514/454

(57) ABSTRACT

The present invention provides for methods and compositions for using cannabinoid compounds, such as ajulemic acid, for the treatment of disorders involving peroxisome proliferator-activated receptor-gamma (PPARγ). In particular, the invention provides for methods and compositions for treating disorders, such as autoimmune or inflammatory disorders, involving PPARγ, such as rheumatoid arthritis and diabetes.

### CROSS REFERENCE TO RELATED APPLICATIONS

This application claims priority from U.S. Provisional Patent Application No. 60/435,184, filed on Dec. 19, 2002, which is incorporated herein by reference in its entirety.

### STATEMENT AS TO FEDERALLY SPONSORED RESEARCH

This invention was made with support from grant numbers DA12178, AR38501, DK52542 and DK52888 from the National Institute of Health. The government has certain rights in the invention.

### FIELD OF THE INVENTION

This application relates to autoimmune agents, and, in particular, to the use of cannabinoids for the treatment of disorders, such as autoimmune and inflammatory disorders, such as diabetes mellitus, and to medicinal preparations containing cannabinoids such as ajulemic acid.

### BACKGROUND OF THE INVENTION

Cannabis sativa contains a group of biosynthetically related substances known collectively as cannabinoids. (F Grotenhermen and E Russo in “Cannabis and Cannabinoids”, Haworth Press, New York (2002) pages xxvii-xxxi). The identification of an endogenous cannabinoid anandamide (Devane, Trends Pharmacol Sci 15: 40-1, 1994), a metabolite of the arachidonic acid that, among other actions, possesses anti-inflammatory activity, has generated a new wave of interest in this area. Tetrahydrocannabinol (THC), one of the major cannabinoids in Cannabis, is known to possess a number of activities such as analgesia and anti-inflammatory action. However, it also has certain undesirable effects on mood and behavior, the so-called psychotropic effects, which limits the clinical application of THC and Cannabis. Considerable effort has been expended toward the goal of discovering non-psychotropic cannabinoid analogs or derivatives that retain certain beneficial actions, but are free of psychotropic activity.

A useful template molecule is the cannabinoid, tetrahydrocannabinol-11-oic (THC-11-oic) acid, a metabolite of THC, because it does not show changes in mood or behavior in human or in animal models. However, it has only modest analgesic and anti-inflammatory activity (Burstein et al., J Med Chem 35:3135-41, 1992). It is known that manipulation of the side-chain structure of THC could produce significant modification of its activities. In particular, increasing the side-chain to seven carbon atoms and the introduction of methyl groups dramatically increases its potency. Based on the same analogy, manipulation of the side chain of THC-11-oic acid resulted in the production of ajulemic acid (AJA), a synthetic analog of THC-11-oic acid (FIG. 1; Burstein et al., J Med Chem 35:3135-41, 1992).

AJA has been shown to posses prolonged analgesic activity (Burstein et al., J Med Chem 35:3135-41, 1992; Dajani et al., J Pharmacol Exp Ther 291:31-8, 1999), and anti-inflammatory activity (Burstein et al., J Med Chem 35:3135-41, 1992; Zurier et al., Arthritis Rheum 41:163-70, 1998). In contrast to the non steroidal anti-inflammatory drugs (NSAIDs), it is non-ulcerogenic at therapeutically relevant doses. Most importantly it shows none of the typical psychotropic actions of THC in animal models, as well as in humans.

Peroxisome proliferator-activated receptors (PPARs) are transducer proteins belonging to the steroid/thyroid/retinoid receptor superfamily. Three subtypes of PPAR have been identified, designated as PPAR-alpha (PPAR-α), PPAR-beta (PPAR-β, or PPAR-delta (PPAR-δ)), and PPAR-gamma (PPAR-γ). PPARγ plays a role in adipocyte differentiation, lipid metabolism, and glucose homeostasis, as well as in modulating anti-inflammatory (Jiang et al., Nature 391:82-6, 1998), and anti-tumor processes (Patel et al., Curr Biol 11:764-8, 2001). Activation of PPARγ can inhibit the expression of cytokines, such as interleukin-1β (IL-1β), tumor necrosis factor α (TNFα), nitric oxide (NO), at both protein and transcription levels (Jiang et al., Nature 391:82-86, 1998; Ricote et al., Nature 391:79-82, 1998). PPARγ agonist is expressed in adipose tissue, skeletal muscle, adrenal gland, colonic epithelium, heart, pancreas, and liver (Mukheriee et al., J Biol Chem 272:8071-6, 1997; Sarraf et al., Nat Med 4:1046-52, 1998. It is also expressed in immune system related cells such as splenocytes (Clark et al., J Immunol 164:1364-71, 2000; Kliewer et al., Proc Natl Acad Sci USA 91:7355-9, 1994), synoviocytes (Ji et al., J Autoimmun 17:215-21, 2001); Kawahito et al., J Clin Invest 106:189-97, 2000; Simonin et al., Am J Physiol Cell Physiol 282:C125-33, 2002), helper T cells (Clark et al., J Immunol 164:1364-71, 2000) and activated monocytes and macrophages (Jiang et al., Nature 391:82-6, 1998; Kawahito et al., J Clin Invest 106:189-97, 2000; Ricote et al., Nature 391:79-82, 1998).

### SUMMARY OF THE INVENTION

The present invention relates to THC cannabinoid compounds, e.g., ajulemic acid (AJA, dimethylheptyl-THC-11-oic acid), pharmaceutical compositions containing them, and methods for using the cannabinoids, e.g., ajulemic acid, as medicaments. More specifically, cannabinoids, e.g., ajulemic acid, of the invention can be utilized in the modification, amelioration, reduction, or prevention of disorders associated with peroxisome proliferator-activated receptors (PPAR), in particular peroxisome proliferator-activated receptor-gamma (PPARγ), and are useful for the treatment of such disorders, including autoimmune, inflammatory, and other disorders, such as diabetes and rheumatoid arthritis.

The present invention is based, in part, on the discovery that autoimmune, inflammatory, and other disorders can be treated or ameliorated by using cannabinoids that effect PPAR activity, e.g., by using a cannabinoid with Formula I (see below) to bind to, and activate PPARγ. Accordingly, in one aspect, the invention pertains to a method for treating a disorder associated with peroxisome proliferator-activated receptor (PPAR) function by administering to a subject a pharmaceutically effective amount of a composition including a cannabinoid compound. The cannabinoid can be combined with an existing anti-inflammatory agent, or anti-diabetic agent. In one embodiment, the cannabinoid is that with “Formula I”, e.g., ajulemic acid:

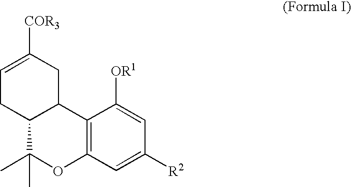

in which R¹ can be a hydrogen atom, —COCH₃, —COCH₂CH₃, or —CO(CH₂)_{N}CH₃ (where N=0-20) and R² can be a branched C₅-C₁₂ alkyl. In certain embodiments, R¹ can be hydrogen and/or R² can be a C₉alkyl, e.g., a branched alkyl, such as 1,1-dimethylheptyl. R₃ can be —OH, —OCH₃, or —NHCH₂COOH. Other cannabinoids include all of those listed in U.S. Pat. Nos. 4,847,290, 4,973,603, 5,338,753, 5,538,993, 5,635,530, 6,162,829, and 6,448,288, which are all hereby incorporated by reference in their entireties.

The cannabinoid can be used to ameliorate or treat a disorder associated with a PPARγ (e.g., an autoimmune disorder (e.g., diabetes mellitus, impaired glucose intolerance, diabetic retinopathy, obesity, systemic lupus erythematosis, rheumatoid arthritis, spondylo arthritis, asthma, inflammatory bowel disease, vasculitis, dermatomyositis, polymyositis, sjogren's syndrome, hyperthyroidism), ankylosing spondylitis, gout, arthritis associated with gout, osteoarthritis, osteoporosis, atherosclerosis, hypertension, hyperglycemia, coronary artery disease, dyslipidemia, exudative age-related macular degeneration, aereolar age-related macular degeneration, atherosclerosis, an inflammatory disorder (e.g., rheumatoid arthritis, multiple sclerosis, myasthenia gravis, uveoretinitis, uveitis, iritis, cyclitis, choroiditis, chorioretinitis, vitritis, keratitis, conjunctivitis, psoriasis, eczema, thyroiditis, a collagen vascular disorder (e.g., ankylosing spondylitis, rheumatoid arthritis, lupus erythematosus, Reiter syndrome, Bechet disease, ulcerative colitis, Crohn's disease, or Wegener's granulomatosis))). For example, the disorder can also be a non-autoimmune inflammatory disorder, such as osteoarthritis. In certain embodiments, for example, the disorder can be diabetes, which can be treated by administering to a subject with diabetes a pharmaceutically effective amount of cannabinoid of Formula I, e.g., ajulemic acid. As a further example, the disorder can be an inflammatory disease associated with PPARγ, such as rheumatoid arthritis, multiple sclerosis, myasthenia gravis, or uveoretinitis.

In another aspect, the invention features methods of modifying or treating an autoimmune disorder associated with peroxisome proliferator-activated receptor gamma (PPARγ) function, including administering to a subject in need thereof an amount of a PPARγ activator effective to modify or ameliorate an autoimmune disorder, wherein the PPARγ activator is a cannabinoid. In certain embodiments, the autoimmune disease is diabetes and can be treated in a subject sensitive to the cannabinoid of Formula I by identifying a subject having diabetes sensitive to Formula I; and administering to the subject an effective amount of a compound of Formula I. Formula I can be administered in a composition orally, systemically, via an implant, for example an implant that provides slow release of the compound, or may also be administered intravenously. The compound can be administered at about 0.1 to 50 mg/kg body weight of the subject, e.g., a mammalian subject, and preferably about 0.2 to 2 mg/kg body weight of the mammalian subject.

In another aspect, the invention features methods of treating a subject (e.g., one sensitive to a cannabinoid) having a disorder (e.g., an autoimmune disorder (e.g., diabetes mellitus, impaired glucose intolerance, diabetic retinopathy, obesity, systemic lupus erythematosis, rheumatoid arthritis, spondylo arthritis, asthma, inflammatory bowel disease, vasculitis, dermatomyositis, polymyositis, sjogren's syndrome, hyperthyroidism), ankylosing spondylitis, gout, arthritis associated with gout, osteoarthritis, osteoporosis, atherosclerosis, hypertension, hyperglycemia, coronary artery disease, dyslipidemia, exudative age-related macular degeneration, aereolar age-related macular degeneration, atherosclerosis, an inflammatory disorder (e.g., rheumatoid arthritis, multiple sclerosis, myasthenia gravis, uveoretinitis, uveitis, iritis, cyclitis, choroiditis, chorioretinitis, vitritis, keratitis, conjunctivitis, psoriasis, eczema, thyroiditis, a collagen vascular disorder (e.g., ankylosing spondylitis, rheumatoid arthritis, lupus erythematosus, Reiter syndrome, Bechet disease, ulcerative colitis, Crohn's disease, or Wegener's granulomatosis))) associated with peroxisome proliferator-activated receptor-gamma (PPARγ) function, in which the method includes administering to the subject a pharmaceutically effective amount of a composition including a cannabinoid compound (e.g., a cannabinoid of Formula I (e.g., in which R² is a C₉ alkyl, a branched alkyl, 1,1-dimethylheptyl), ajulemic acid). In these methods, the dosage of the cannabinoid can be between about 0.1 and 50 mg/kg body weight of the subject, and the mode of administration of the cannabinoid can be oral, nasal, pulmonary, transdermal, or parenteral. The subject can be a human or an animal (e.g., a mammal (e.g., non-human primate, dog, cat, rodent, horse, cow, sheep, or goat)).

In another aspect, the invention features kits comprising a pharmaceutically effective amount of a composition including a cannabinoid compound (e.g., a cannabinoid of Formula I (e.g., in which R² is a C₉ alkyl, a branched alkyl, 1,1-dimethylheptyl), ajulemic acid), as well as instructions for use of the composition in treating a subject (e.g., one sensitive to a cannabinoid) having a disorder (e.g., an autoimmune disorder (e.g., diabetes mellitus, impaired glucose intolerance, diabetic retinopathy, obesity, systemic lupus erythematosis, rheumatoid arthritis, spondylo arthritis, asthma, inflammatory bowel disease, vasculitis, dermatomyositis, polymyositis, sjogren's syndrome, hyperthyroidism), ankylosing spondylitis, gout, arthritis associated with gout, osteoarthritis, osteoporosis, atherosclerosis, hypertension, hyperglycemia, coronary artery disease, dyslipidemia, exudative age-related macular degeneration, aereolar age-related macular degeneration, atherosclerosis, an inflammatory disorder (e.g., rheumatoid arthritis, multiple sclerosis, myasthenia gravis, uveoretinitis, uveitis, iritis, cyclitis, choroiditis, chorioretinitis, vitritis, keratitis, conjunctivitis, psoriasis, eczema, thyroiditis, a collagen vascular disorder (e.g., ankylosing spondylitis, rheumatoid arthritis, lupus erythematosus, Reiter syndrome, Bechet disease, ulcerative colitis, Crohn's disease, or Wegener's granulomatosis))) associated with peroxisome proliferator-activated receptor-gamma (PPARγ) function.

The invention also includes compositions for use as medicaments in treating a subject (e.g., one sensitive to a cannabinoid) having a disorder (e.g., an autoimmune disorder (e.g., diabetes mellitus, impaired glucose intolerance, diabetic retinopathy, obesity, systemic lupus erythematosis, rheumatoid arthritis, spondylo arthritis, asthma, inflammatory bowel disease, vasculitis, dermatomyositis, polymyositis, sjogren's syndrome, hyperthyroidism), ankylosing spondylitis, gout, arthritis associated with gout, osteoarthritis, osteoporosis, atherosclerosis, hypertension, hyperglycemia, coronary artery disease, dyslipidemia, exudative age-related macular degeneration, aereolar age-related macular degeneration, atherosclerosis, an inflammatory disorder (e.g., rheumatoid arthritis, multiple sclerosis, myasthenia gravis, uveoretinitis, uveitis, iritis, cyclitis, choroiditis, chorioretinitis, vitritis, keratitis, conjunctivitis, psoriasis, eczema, thyroiditis, a collagen vascular disorder (e.g., ankylosing spondylitis, rheumatoid arthritis, lupus erythematosus, Reiter syndrome, Bechet disease, ulcerative colitis, Crohn's disease, or Wegener's granulomatosis))) associated with peroxisome proliferator-activated receptor-gamma (PPARγ) function, in which the compositions include a pharmaceutically effective amount of a cannabinoid compound or PPARγ activator (e.g., a cannabinoid of Formula I (e.g., in which R² is a C₉ alkyl, a branched alkyl, 1,1-dimethylheptyl), ajulemic acid). With respect to these compositions, the dosage of the cannabinoid can be between about 0.1 and 50 mg/kg body weight of the subject, and the mode of administration of the cannabinoid can be oral, nasal, pulmonary, transdermal, or parenteral. The subject can be a human or an animal (e.g., a mammal (e.g., non-human primate, dog, cat, rodent, horse, cow, sheep, or goat)).

In addition, the invention encompasses uses of compositions for the manufacture of a medicament for use in treating a subject (e.g., one sensitive to a cannabinoid) having a disorder (e.g., an autoimmune disorder (e.g., diabetes mellitus, impaired glucose intolerance, diabetic retinopathy, obesity, systemic lupus erythematosis, rheumatoid arthritis, spondylo arthritis, asthma, inflammatory bowel disease, vasculitis, dermatomyositis, polymyositis, sjogren's syndrome, hyperthyroidism), ankylosing spondylitis, gout, arthritis associated with gout, osteoarthritis, osteoporosis, atherosclerosis, hypertension, hyperglycemia, coronary artery disease, dyslipidemia, exudative age-related macular degeneration, aereolar age-related macular degeneration, atherosclerosis, an inflammatory disorder (e.g., rheumatoid arthritis, multiple sclerosis, myasthenia gravis, uveoretinitis, uveitis, iritis, cyclitis, choroiditis, chorioretinitis, vitritis, keratitis, conjunctivitis, psoriasis, eczema, thyroiditis, a collagen vascular disorder (e.g., ankylosing spondylitis, rheumatoid arthritis, lupus erythematosus, Reiter syndrome, Bechet disease, ulcerative colitis, Crohn's disease, or Wegener's granulomatosis))) associated with peroxisome proliferator-activated receptor-gamma (PPARγ) function, in which the compositions include a pharmaceutically effective amount of a cannabinoid compound or PPARγ activator (e.g., a cannabinoid of Formula I (e.g., in which R² is a C₉ alkyl, a branched alkyl, 1,1-dimethylheptyl), ajulemic acid). In these uses, the dosage of the cannabinoid can be between about 0.1 and 50 mg/kg body weight of the subject, and the mode of administration of the cannabinoid can be oral, nasal, pulmonary, transdermal, or parenteral. The subject can be a human or an animal (e.g., a mammal (e.g., non-human primate, dog, cat, rodent, horse, cow, sheep, or goat)).

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

The term “sample” as used herein refers to a test item that has a component that is capable of interacting with a cannabinoid, e.g., a biological sample with PPARγ that can interact with AJA. The sample can be a liquid or fluid biological sample, or a solid biological sample. The biological sample can be a liquid sample e.g., blood, plasma, serum, cerebral spinal fluid, urine, amniotic fluid, interstitial fluid, and synovial fluid. The sample may be a solid, e.g., a tissue or cell matter. The term “sample” also refers to a non-biological sample such as a chemical solution, or synthetic composition. In one embodiment, the sample is blood. In another embodiment, the sample is plasma.

The term “peroxisome proliferator-activated receptor” and “PPAR” are art recognized term referring to a family of transducer proteins belonging to the steroid/thyroid/retinoid receptor superfamily. PPARs are described in a review article by Schoonjans (1996) J. Lipid Res. 37:907-925, and described in more detail below. Three subtypes of PPAR have been identified, and these are designated as alpha (α), beta (β) (or delta (δ)), and gamma (γ). In a preferred embodiment, cannabinoids, such as AJA bind to, and activate PPARγ.

The term “PPARγ” as used herein refers to all isotypes of PPARγ. PPARγ exists as at least two isotypes, PPARγ1 and PPARγ2. The term “PPARγ” refers to any of these isotypes or combination thereof.

The phrase “PPARγ associated disorder” as used herein refers to a pathological condition in a subject that results at least, in part, from PPARγ function. The phrase “PPARγ associated disorder” is also intended to include any disorders in which the manifestation of the disorder is characterized by the disturbance in the regulation of mood, behavior, control of feeding behavior. Particularly preferred PPARγ associated disorder include autoimmune disorders that involve PPARγ. Examples or autoimmune diseases include, but are not limited to, rheumatoid arthritis, diabetes mellitus, glucose intolerance.

The term “modifies” or “modified” are used interchangeably herein and refer to the up-regulation, or down-regulation, or activation of the target PPAR, e.g., PPARγ. The term “modifies” or “modified” also refers to the increase, decrease, elevation, or depression of processes or signal transduction cascades involving a target PPAR, e.g., PPARγ. Modification to the PPARγ may occur when a cannabinoid, e.g., AJA, binds to the PPARγ. This modification may directly affect the PPARγ, for example modifications that may result in an increase in PPARγ number. Alternatively, the modifications may occur as an indirect effect of binding to the PPARγ. For example, binding of AJA to the PPARγ can also lead to a change in downstream processes involving the PPARγ, such as the recruitment of co-activators by the PPARγ-AJA complex. In addition, antagonism of transcription factors can facilitate gene expression of mediators of inflammation, thereby reducing production of the mediators. The modifications can therefore be direct modifications of the PPARγ, or an indirect modification of a process or cascade involving the PPARγ. Non-limiting examples of modifications includes modifications of morphological and functional processes, under- or over production or expression of substances, induction of differentiation of cells, e.g., induction of adipocyte differentiation, and recruitment of co-activators, e.g., DRIP205.

The terms “modify” and “modified” also include treating a subject prophylactically to alter inflammation, apoptosis, proliferation, autoimmune function, and expression of oncogenes and other genes controlling cell metabolism. The present methods include both medical therapeutic and/or prophylactic treatment, as necessary.

The term “cannabinoid” as used herein refers to biosynthetically related compounds such as delta-8-tetrahydrocannabinol, delta-9-tetrahydrocannabinol, cannabidol, olivetol, cannabinol, cannabigerol, nabilone, delta-9-tetrahydro cannabinol-11 oic acid. The non-psychotropic cannabinoid 3-dimethylheptyl 11 carboxylic acid homolog of, delta-8-tetrahydrocannabinol (Burstein et al. J. Med. Chem. (1992) 35: 3135). A useful cannabinoid is ajulemic acid (AJA; dimethylheptyl-THC-11-oic acid). The term “cannabinoid” also includes the cannabinoid compounds, their derivatives, analogs, tautomeric forms, stereoisomers, polymorphs, metabolic analogues, pharmaceutically acceptable salts, pharmaceutically acceptable solvates, and pharmaceutical compositions containing them. The compounds are useful in the treatment of diabetes mellitus (Type 1 and Type II), impaired glucose tolerance, insulin resistance, obesity, and other diseases. The AJA acting through PPARγ can also be used to treat or modify autoimmune diseases such as rheumatoid arthritis.

The term “activator” is used to denote any molecular species that results in activation of a PPAR, e.g., PPARγ regardless of whether the species itself binds to the receptor or a metabolite of the species binds to the receptor.

The term “PPARγ activator” as used herein refers to a molecular species that activates PPAR-gamma.

The term “alkyl” as used herein refers to a straight or branched hydrocarbon chain containing carbon atoms or cyclic hydrocarbon moieties. These alkyl groups may also contain one or more double bonds or triple bonds. By “substituted alkyl” is meant an alkyl in which an atom of the alkyl is substituted with an atom, e.g., a sulfur, oxygen, or halogen atom.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a series of representations of the chemical structures of THC, THC-11-oic acid, and ajulemic acid.

FIG. 2A is a representation of a membrane showing partial protease digestion assays indicating that AJA binds to PPARγ. AJA induces PPARγ to a conformation that is more resistant to trypsin digestion. AJA treatment protects mPPARγ from trypsin digestion.

FIG. 2B is a representation of a membrane showing partial protease digestion assays indicating that AJA binds to PPARγ. The resistance to trypsin digestion was positively related to the concentration of AJA in a dose response manner.

FIG. 2C is a representation of a membrane showing partial protease digestion assays in which AJA provides concentration-dependent protection of mPPARγ against trypsin digestion.

FIG. 2D is a representation of a membrane showing partial protease digestion assays indicating that PPARδ was digested evenly when treated with different concentrations of AJA.

FIG. 3A is a bar graph showing the activation of PPARγ by GW347845 (GW34) and AJA in a concentration-dependent manner.

FIG. 3B is a bar graph showing that PPARα was not activated by 20 μM of AJA in a reporter gene assay.

FIG. 3C is a bar graph showing that PPARδ was not activated by 20 μM of AJA in a reporter gene assay.

FIG. 3D is a bar graph showing that AJA does not activate RXRα. Only 9-cis RA activated RXRα.

FIG. 3E is a bar graph showing that the AF-2 helix truncated PPARγ cannot be activated by AJA or GW34. The activation of PPARγ requires AF-2 helix of the receptor.

FIG. 4A is a schematic diagram of the GAL4 based reporter system showing that AJA activates PPARγ in a heterologous reporter system. The PPARγ was expressed as a Gal4 DBD fusion protein, which binds to the UAS promoter containing 4 copies of the GAL4 binding sites upstream of the minimal TK promoter. Binding of PPARγ activators, such as AJA, activates the luciferase reporter gene expression.

FIG. 4B is a bar graph showing that PPARγ was significantly activated by 20 μM of AJA or 1 μM of GW34 that served as a positive control.

FIG. 4C is a graph showing the dose-dependent activation of PPARγ by AJA by measuring luciferase activity.

FIG. 4D is a graph showing the fold activation of human PPARγ by AJA , while AJA failed to activate PPARα and PPARδ, and PPARγ.

FIG. 5A is a photograph of a membrane showing the interaction of PPARγ with coactivator GST-DRIP205 (amino acids 527-970) in the presence of GW34 or AJA.

FIG. 5B is a photograph of a membrane showing the interaction of PPARγ with coactivator GST-RAC3 (amino acids 613-752) in the presence of GW34 or AJA.

FIG. 6A is a bar graph of PPARγ transfected cells in which AJA reduces the PMA-activated IL-8 promoter activity in a concentration (in μM)-dependent manner.

FIG. 6B is a bar graph showing that AJA had no effect on IL-8 promoter activity in the PPARγ ΔAF2 transfected cells.

FIG. 6C is a bar graph showing that GW347845 reduces the IL-8 promoter activity in cells transfected with wild type PPARγ.

FIG. 6D is a bar graph showing that GW347845 had no effect on IL-8 promotor activity in cells transfected with the PPARγ ΔAF2 mutant.

FIGS. 7A-C are representations of 3T3 L1 cells. FIG. 7A depicts treatment with 0.1% DMSO control (vehicle). FIG. 7B depicts treatment with 1 μM of GW347845 in 0.1% DMSO (GW34). FIG. 7C depicts treatment with 20 μM of AJA in 0.1% DMSO (AJA). The results demonstrate that AJA induces adipocyte differentiation in 3T3 L1 cells; and

FIG. 7D is a representation of a gel showing RT-PCR analysis of adipocyte specific genes.

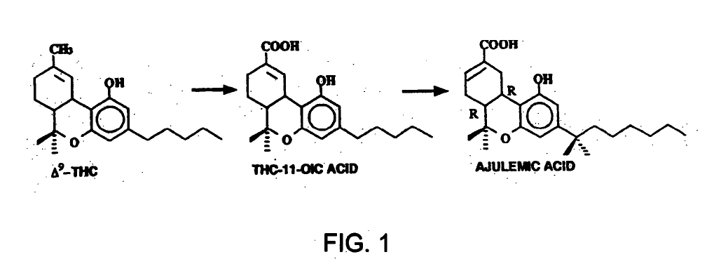

### DETAILED DESCRIPTION OF THE INVENTION

The practice of the present invention employs, unless otherwise indicated, conventional methods of virology, microbiology, molecular biology, and recombinant DNA techniques within the skill of the art. Such techniques are explained fully in the literature (see, e.g., Sambrook, et al. Molecular Cloning: A Laboratory Manual (Current Edition); DNA Cloning: A Practical Approach, Vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbooks).

The invention is based, in part, on the discovery that the cannabinoid with Formula I, e.g., ajulemic acid (AJA, dimethylheptyl-THC-11-oic acid), a synthetic analog of THC-11-oic acid, binds directly and specifically with peroxisome proliferator activated nuclear receptor gamma (PPARγ), and modifies the effect of the receptor. In particular, AJA binding to PPARγ produces an AJA-PPARγ complex that effects down stream processes, for example, recruitment of co-activators such as DRIP205, and antagonism of transcription factors which facilitate gene expression of mediators of inflammation, thereby reducing production of the mediators.

The methods and compositions of the invention can be used to target PPARγ for therapeutic effects of AJA on autoimmune diseases or disorders that involve PPARγ, such as diabetes, insulin resistance, glucose intolerance, NIDDM, and lipid metabolism. The identification of PPARγ as a selective target for AJA allows development of more effective drugs for the treatment of diabetes and obesity, as well as other autoimmune diseases involving PPARγ.

The invention is described in more detail in the following subsections.

I. PPAR Family In one aspect, the invention features using THC cannabinoids of Formula I, such as ajulemic acid (AJA), as activators of a peroxisome proliferator-activated receptor (PPAR) family member, in particular, the activation of PPARγ. The PPARs are transducer proteins belonging to the steroid/thyroid/retinoid receptor superfamily. Three subtypes of PPAR have been identified, designated as PPAR-alpha (PPAR-α), PPAR-beta (PPAR-β or PPAR-delta (PPAR-δ)), and PPAR-gamma (PPAR-γ). These receptors function as activator-regulated transcription factors that control the expression of target genes by binding to their responsive DNA sequence as heterodimers with retinoid x receptor (RXR).

The PPARα subtype has been cloned from Xenopus, humans, mouse, and rat; the PPARβ (or PPARδ) subtype from Xenopus, humans, and mouse; and the PPARγ subtype from Xenopus, humans, and hamster. These subtypes are pharmacologically distinct and differentially activated by various agents (Yu et al., Cell 67:1251-1266, 1991. The following are corresponding Genbank accession numbers: PPARα(δ) (AF246303), PPARβ (AL022721), and PPARγ (AY157024). PPARγ exists as at least two isotypes, PPARγ1 and PPARγ2. PPARγ2 is expressed selectively in adipose tissue, whereas PPARγ1 is expressed at lower levels in a variety of other rodent and human tissues (Spiegelman, Diabetes 47:507-514, 1998).

PPARγ is a pharmacologically important member of the nuclear receptor superfamily (Houseknecht et al., Domest Anim Endocrinol 22:1-23, 2002). It plays important roles in a diverse array of biological processes including lipid metabolism, glucose homeostasis, and adipocyte differentiation. The crystal structure of the PPARγ ligand-binding domain reveals a large hydrophobic cavity for ligand binding (Uppenberg et al., J Biol Chem 273:31108-12, 1998; and Xu et al., Proc Natl Acad Sci USA 98:13919-24,2001). Indeed, PPARγ binds to a wide range of synthetic and naturally occurring substances, including the antidiabetic drugs thiazolidinediones (Lehmann et al., J Biol Chem 270:12953-6, 1995; Willson et al., J Med Chem 39:665-8, 1996), the synthetic tyrosine analog GW347845 (Cobb et al., J Med Chem 41:5055-5069, 1998), polyunsaturated fatty acids (Kliewer et al., Proc Natl Acad Sci USA 94:4318-23, 1997), metabolites of arachidonic acid including 15-deoxy-Δ^{12,14}prostaglandin J₂ (Forman et al., Cell 83:803-12, 1995; Kliewer et al, Cell 83:813-9, 1995), NSAIDs (Lehmann et al., J Biol Chem 272:3406-10, 1997), and compounds of oxidized low-density lipoprotein, such as 13-hydroxyoctadecadienoic acid (13-HODE) and 15-hydroxyeicosatraenoic acid (15-HETE) (Nagy et al., Cell 93:229-40, 1998). Several of these PPARγ ligands exhibit antiinflammatory activity in vivo (Kawahito et al., J Clin Invest 106:189-97, 2000; Naito et al., Aliment Pharmacol Ther 15:865-73, 2001), and activation of PPARγ is directly linked to antiinflammatory (Jiang et al., Nature 391:82-6, 1998), and antitumor processes (Patel et al., Curr Biol 11:764-8, 2001). Accordingly, activation of PPARγ inhibits the expression of cytokines-, such as interleukin (IL)-1 β, tumor necrosis factor α (TNFα), and nitric oxide (NO) at both the protein and transcription levels (Jiang et al., Nature 391:82-6, 1998; Ricote et al., Nature 391:79-82, 1998). PPARγ is expressed in adipose tissue, skeletal muscle, adrenal gland, colonic epithelium, heart, pancreas, and liver (Mukherjee et al., J Biol Chem 272:8071-6, 1997; Sarraf et al., Nat Med 4:1046-52, 1998). It is also expressed in immune system related cells such as splenocytes (Clark et al., J Immunol 164:1364-71, 2000; Kliewer et al., Proc Natl Acad Sci USA 91:7355-9, 1994), synoviocytes (Ji et al., J Autoimmun 17:215-21, 2001; Kawahito et al., J Clin Invest 106:189-97, 2000; Simonin et al., Am J Physiol Cell Physiol 282:C125-33, 2002), helper T cells (Clark et al., J Immunol 164:1364-71, 2000), and activated monocytes and macrophages (Jiang et al., Nature 391:82-6, 1998; Kawahito et al., J Clin Invest 106:189-97, 2000; Ricote et al., Nature 391:79-82, 1998) suggesting that PPARγ has a direct role in modulating inflammation in addition to its role in lipid metabolism and glucose homeostasis.

PPARγ modulates the expression of genes involved in the regulation of growth and differentiation in a variety of cell types that express the receptor. PPARγ has been shown to be expressed in an adipose tissue-specific manner. Its expression is induced early during the course of differentiation of several preadipocyte cell lines. PPARγ plays a role in the adipogenic signaling cascade and also regulates the ob/leptin gene which is involved in regulating energy homeostasis.

It was determined in the present invention that AJA is an activator of PPARγ and binds to PPARγ directly (see Examples). The data demonstrates that activation of PPARγ by AJA may contribute to the analgesic and anti-inflammatory actions of AJA. These data also demonstrate that AJA may have other biological functions, since PPARγ regulates a wide range of cellular activities.

Treatment of peritoneal macrophages with 15d-PGJ2 or several synthetic PPARγ activators suppressed the expression of the inducible nitric oxide synthase (iNOS) and inhibited induction of gelatinase B and scavenger receptor, a gene induced by phorbol ester stimulation. The promoters of these genes were found to possess binding sites for activator protein-1 (AP-1), nuclear factor κB (NF-κB), and signal transducer and activator of transcription (STAT). Furthermore, the inhibition of the inflammatory response in macrophages was found in part by antagonizing the activities of these transcription factors (Ricote et al., Nature 391, 1998).

Because PPARγ forms a permissive heterodimer with RXR in vivo, activators for both receptors were shown to inhibit the LPS-induced NO and TNF-α production and the combined treatment with the two activators resulted in synergistic inhibition. Activation of PPARγ/RXR did not affect the translocation of NF-κB to nucleus, NF-κB activation in (EMSA), or the phosphorylation of JNK on LPS stimulation, while in the meantime it suppressed LPS-activated NF-κB promoter activity. This indicates that the inhibition was achieved at the transcription level (Uchimura et al., Hepatology 33:91-9, 2001). Even though the promoter regions of the iNOS and TNF-α contain binding sites for NF-κB and AP-1, they do not contain consensus PPAR response elements, indicative that it is unlikely that PPARγ binds to, or controls these promotors directly. The inhibition of inflammatory response by activation of PPARγ was confirmed by recent studies both in vitro (Ji et al., J Autoimmun 17:215-21, 2001) and in vivo (Dubuquoy et al., Gastroenterol Clin Biol 24:719-24, 2000; Kawahito et al., J Clin Invest 106:189-97, 2000; Naito et al., Aliment Pharmacol Ther 15:865-73, 2001).

In these studies, the anti-inflammatory effects of 15d-PGJ2 were shown to be more potent than any synthetic PPARγ activator. It has been demonstrated that 15d-PGJ2 and troglitazone modulated the expressions of LPS-induced iNOS, COX-2, and pro-inflammatory cytokines differently. 15d-PGJ2 suppressed the expressions of iNOS, COX-2, IL-1β and TNFα. Troglitazone only inhibited the iNOS, and TNF α expressions, indicating that additional mechanisms may be involved in 15d-PGJ2-mediated anti-inflammatory effect (Simonin et al., Am J Physiol Cell Physiol 282:C125-33, 2002).

Recent studies demonstrated that 15d-PGJ2 also mediated anti-inflammatory action through PPARγ-independent manner. The 15d-PGJ2 suppressed the IL-1β-induced PGE₂ synthesis by inhibiting the expressions of COX-2 and cytosolic phospholiphase A₂ (cPLA₂), which are critical enzymes during the synthesis of prostaglandins (Tsubouchi et al., Biochem Biophys Res Commun 283:750-5, 2001). In resting cells, NF-κB is sequestrated in the cytoplasm by association with an inhibitory protein IκB. In response to the signaling from inflammatory cytokine, the IκB kinase (IKK) is activated and phosphorylates IκB, such that it is readily digested by proteasome, allowing NF-κB to migrate into the nucleus and activate gene expression (Ghosh et al., Annu Rev Immunol 16:225-60, 1998). 15d-PGJ2 was shown to inhibit IKK (Straus et al., Proc Natl Acad Sci USA 97:4844-9, 2000) and directly inhibit DNA binding of NF-κB (Straus et al., Proc Natl Acad Sci USA 97:4844-9, 2000; Simonin et al., Am J Physiol Cell Physiol 282:C125-33, 2002).

The activator binding domain of PPARγ is much larger than the other nuclear receptor so that PPARγ appears to bind a range of synthetic and naturally occurring substances, including antidiabetic drug thiazolidinediones (Lehmann et al., J Biol Chem 270:12953-6, 1995; Willson et al., J Med Chem 39:665-8, 1996), polyunsaturated fatty acid (Kliewer et al., Proc Natl Acad Sci USA 94:4318-23, 1997), 15-deoxy-Δ^{12,14}prostaglandin J₂ (Forman et al., Cell 83:803-12, 1995; Kliewer et al., Cell 83:813-9, 1995), NSAIDS (Lehmann et al., J Biol Chem 272:3406-10, 1997), and compounds of oxidized low-density lipoprotein, such as 13-hydroxyoctadecadienoic acid (13-HODE) and 15-hydroxyeicosatrtraenoic acid (15-HETE) (Nagy et al., Cell 93:229-40, 1998).

II. Cannabinoids and Cannabinoid Receptors

In one aspect, the invention pertains to using THC cannabinoids, and derivatives of cannabinoids such as ajulemic acid, for modifying a disorder associated with PPARγ. The term “cannabinoids” refers to organic substances present in Cannabis sativa, having a variety of pharmacological properties.

Ajulemic acid (AJA, dimethylheptyl-THC-11-oic acid) is a non-psychoactive, synthetic cannabinoid based on the template of THC-11-oic acid. THC is biotransformed by oxidation to its principal metabolite, THC-11-oic acid. AJA is synthesized by increasing the side chain of THC-11-oic acid from 5 carbon atoms (pentyl) to 7 atoms (heptyl) and by introducing 2 methyl groups.

AJA has been shown to produce potent and prolonged analgesic effects (Burstein et al, Life Sci 63:161-8, 1998; Dajani et al., J Pharmacol Exp Ther 291:31-8, 1999), and anti-inflammatory function (Burstein et al., J Med Chem 35:3135-41, 1992; Zurier et al., Arthritis Rheum 41:163-70, 1998). In the mouse tail clip assay for analgesia, AJA was shown to exert a potent analgesic function comparable with morphine and the effect lasted even longer than morphin (Dajani et al., J Pharmacol Exp Ther 291:31-8, 1999). AJA was also shown to suppress both acute inflammation induced by injection of IL-1β and TNFα into subcutaneous air pouches in mice, and chronic joint inflammation of adjuvant-induced poly-arthritis in rats (Zurier et al., Arthritis Rheum 41:163-70, 1998). In contrast to NSAIDs, it was totally non-ulcerogenic at therapeutically relevant doses and did not exhibit drug dependence or cause mutagenesis (Dajani et al., J Pharmacol Exp Ther 291:31-8, 1999; Burstein, Curr Pharm Des 6:1339-45, 2000). Most importantly, AJA does not show the typical psychoactive effect in animal models (Burstein et al., J Med Chem 35:3135-41, 1992). The mechanism of AJA function is still not fully understood. Its stereospecificity suggested that its functions might be receptor mediated. Two receptors for cannabinoids, cannabinoid receptor-1 (CB1) and CB2, have been identified and cloned (Pertwee, Pharmacol Ther 74:129-80, 1997).

A recent study demonstrated that AJA exhibits modest affinity to CB2, and was found to have CB2-mediated anti-tumor effects (Recht et al., Biochem Pharmacol 62:755-63, 2001). This finding is consistent with the lack of psychoactivity in AJA because, unlike the CB1 receptor, which is present in brain and peripheral tissue, the CB2 receptor is expressed mainly in extraneural, primarily immune tissue.

AJA was also found to suppress COX-2 activity at 1-10 μM concentration range, while having no effect on COX-1 activity at concentrations between 0.25-25 μM (Zurier et al., Arthritis Rheum 41:163-70, 1998). COX-1 is a constitutive enzyme thought to be responsible for production of eicosanoids that help maintain normal renal function, gastric mucosal integrity, and homeostasis (Griswold et al., Med Res Rev 16:181-206, 1996). COX-2 is expressed during inflammatory reactions such as those induced by cytokines, and is therefore thought to be one of the mediators of inflammation (Arias-Negrete et al., Biochem Biophys Res Commun 208:582-9, 1995). This probably can explain why AJA and cannabinoid acids have no ulcerogenisity, while indomethacin, which inhibits both COX-1 and COX-2, has such side effects (Griswold et al., Med Res Rev 16: 181-206, 1996).

The downregulation of COX-2 by AJA may cause a reduction of prostaglandin synthesis, and thus attenuate the catalepic state induced by PGs (Burstein et al., Experientia 43:402-3, 1987). The properties of AJA, including anti-inflammation, anti-tumor effect, and in particular, the adipogenesity demonstrated recently (Recht et al., Biochem Pharmacol 62:755-63, 2001), raised the possibility that AJA may mediate its functions in part by activating PPARγ. In line with this hypothesis, some NSAIDs, the traditional anti-inflammatory agents, have been recently demonstrated to be able to activate PPARγ (Lehmann et al., J Biol Chem 272:3406-10, 1997).

Ajulemic acid (AJA) is a non-psychoactive synthetic cannabinoid based on the template of THC-11-oic acid, which is the natural derivative of THC. THC is biotransformed by oxidation to its principal metabolite, THC-11-oic acid. AJA is synthesized by increasing the side chain of THC-11-oic acid from 5 carbon atoms (pentyl) to 7 atoms (heptyl) and by introducing 2 methyl groups.

III. PPAR Activators

In one aspect, the invention pertains to using THC cannabinoids, e.g., AJA as activators of PPAR, e.g., PPARγ. In one embodiment, the cannabinoid has the following structural formula (Formula I):

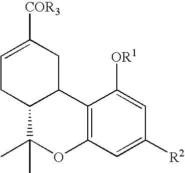

where R¹ is a hydrogen atom, —COCH₃ or —COCH₂CH₃, and R² is a branched C₅-C₁₂ alkyl. R¹ can be hydrogen, and R² can be a C₉ alkyl, which can be a branched alkyl such as 1,1-dimethylheptyl. R₃ is —OH, —OCH₃ or —NHCH₂COOH.

The cannabinoids defined by Formula I have reduced or no psychoactivity and do not bind to the CB1 receptor. Such cannabinoids are known and can be synthesized (see, e.g., U.S. Pat. No. 5,338,753; Burstein et al., J Medicinal Chem. 35:3185-3141, 1992; Burstein, Pharmacol. Ther. 82:87-96, 1999).

Before administration to a subject, the cannabinoids can be tested for biological activity (i.e., ability to decrease cell proliferation) both in vitro or in vivo. In vitro testing can be performed as described in the examples section. In vivo animal models for diabetes are well known in the art, for example, obese-diabetic mice (ob/ob), and obese-diabetic (db/db) mice from the Jackson Laboratories (Bar Harbor, Me)(see, e.g., Collins et al., J Biol Chem 271:9437-9440, 1996; Darling, Curr Opin Genet Dev 6:289-294, 1996; Andersson, Ann. Med. 28:5-7, 1996; Van Heek et al., J. Clin. Invest 99:385-390, 1997). These animal models can be used to assess the effect of the cannabinoids on diabetes and obesity.

The cannabinoid, such as that in Formula I can be administered alone to activate PPARγ, or in addition with existing naturally occurring or synthetic activators to obtain a synergistic effect.

Examples of naturally occurring activators that modify the activity of PPARγ include, but are not limited to, arachidonic acid derivatives or metabolites such as eicosanoids (e.g., various isomeric forms of 8-hydroxytetraenoic acid) and cyclopentenone prostaglandins (e.g., prostaglandins in the J and A series and their metabolites), and polyunsaturated fatty acids.

Examples of synthetic activators that modify the activity of PPARγ include, but are not limited to, antidyslipidemic fibrates (e.g., clofibrate, fenofibrate, benzofibrate, ciprofibrate, gemfibrozil), thiazolidine derivatives (e.g., thiazolidinediones), oxazolidine derivatives (e.g., oxazolidinediones), alpha-alkylthio, alpha-alkoxy and carboxylic acid derivatives of thiazolidines and oxazolidines (Hulin et al., J Med Chem 39:3897-3907, 1996), N-2-L-tyrosine derivatives (Henke et al., J Med Chem; 41:5020-5036, 1998; Collins et al., J Med Chem 41:5037-5054, 1998; Cobb et al., J Med Chem, 41:5055-5069, 1998), phenyl acetic acid derivatives (Berger et al., J Biol. Chem. 274:6718-6725, 1999), and indole-thiazolidinedione derivatives (Lohray et al., J Med Chem, 41:1619-1630, 1998).

IV. Compositions and Formulations

In one aspect, this invention provides methods and compositions for preventing or inhibiting autoimmune diseases or disorders involving PPARγ by using a PPARγ cannabinoid activator, such as ajulemic acid. In general, the methods involve providing an amount of a PPARγ activator sufficient to modulate the expression of genes encoding proteins involved in autoimmune diseases, such as diabetes mellitus, inflammatory cytokines such as tumor necrosis factor-α (TNF α), or inhibition of the production of inflammatory cytokines such as IL-1α, IL-1β, IL-2, IL-6, IL-8, and TNF-α, or the inhibition of their biological activity. While the PPARγ activator can be utilized alone, the therapy can also be utilized in combination with other therapeutics such as existing naturally occurring or synthetic activators, steroidal and non-steroidal anti-inflammatory agents, existing therapies for diabetes, and agents that modulate apoptosis in pathological cells.

In one embodiment of the invention, the cells to be treated are those involved in inflammatory disorders. These include inflammatory (immune system) cells (e.g., T lymphocytes and macrophages), PPARγ expressing cells and tissues involved in the pathogenesis of inflammatory diseases, including all forms of uveitis and uveoretinitis, iritis, cyclitis, choroiditis, chorioretinitis, vitritis, keratitis and conjunctivitis, systemic autoimmune disorders (e.g., type 1 diabetes mellitus, sjogren's syndrome and hyperthyroidism), and collagen vascular diseases (e.g., ankylosing spondylitis, rheumatoid arthritis, lupus erythematosus, Reiter syndrome, Bechet disease, ulcerative colitis, Crohn's disease, Wegener's granulomatosis).

In another embodiment, the cells to be treated are those involved in autoimmune disorders, such as the pancreatic B cells of a subject with diabetes.

Pharmaceutical compositions containing a compound of interest may be prepared by conventional techniques (e.g., as described in Remington: The Science and Practise of Pharmacy, 19th Ed., 1995). The compositions may appear in conventional forms, for example capsules, tablets, aerosols, solutions, suspensions or topical applications.

Typical compositions include the cannabinoid, e.g., AJA, or a derivative thereof, associated with a pharmaceutically acceptable excipient which may be a carrier or a diluent or be diluted by a carrier, or enclosed within a carrier which can be in the form of a capsule, sachet, paper or other container. In making the compositions, conventional techniques for the preparation of pharmaceutical compositions may be used. For example, the compound of interest will usually be mixed with a carrier, or diluted by a carrier, or enclosed within a carrier which may be in the form of an ampule, capsule, sachet, paper, or other container. When the carrier serves as a diluent, it may be solid, semi-solid, or liquid material which acts as a vehicle, excipient, or medium for the active compound. The compound of interest can be adsorbed on a granular solid container for example in a sachet. Some examples of suitable carriers are water, salt solutions, alcohols, polyethylene glycols, polyhydroxyethoxylated castor oil, peanut oil, olive oil, lactose, terra alba, sucrose, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose, and polyvinylpyrrolidone. Similarly, the carrier or diluent may include any sustained release material known in the art, such as glyceryl monostearate or glyceryl distearate, alone or mixed with a wax. The formulations may also include wetting agents, emulsifying and suspending agents, preserving agents, sweetening agents or flavouring agents. The formulations of the invention may be formulated so as to provide quick, sustained, or delayed release of the active ingredient after administration to the patient by employing procedures well known in the art.

The pharmaceutical compositions can be sterilized and mixed, if desired, with auxiliary agents, emulsifiers, salt for influencing osmotic pressure, buffers and/or coloring substances and the like, which do not deleteriously react with the active compounds.

The route of administration may be any route, which effectively transports the compound of interest to the appropriate or desired site of action, such as oral, nasal, pulmonary, transdermal or parenteral, e.g., rectal, depot, subcutaneous, intravenous, intraurethral, intramuscular, intranasal, ophthalmic solution or an ointment, the oral route being preferred.

For nasal administration, the preparation may contain the compound of interest dissolved or suspended in a liquid carrier, in particular an aqueous carrier, for aerosol application. The carrier may contain additives such as solubilizing agents, e.g., propylene glycol, surfactants, absorption enhancers such as lecithin (phosphatidylcholine) or cyclodextrin, or preservatives such as parabens.

To prepare topical formulations, the compound interest is placed in a dermatological vehicle as is known in the art. The amount of the compound of interest to be administered and the compound's concentration in the topical formulations depend upon the vehicle, delivery system or device selected, the clinical condition of the patient, the side effects and the stability of the compound in the formulation. Thus, the physician employs the appropriate preparation containing the appropriate concentration of the compound of interest and selects the amount of formulation administered, depending upon clinical experience with the patient in question or with similar patients.

For ophthalmic applications, the compound of interest is formulated into solutions, suspensions, and ointments appropriate for use in the eye. The concentrations are usually as discussed above for local preparations. For ophthalmic formulations (see Mitra (ed.), Ophthalmic Drug Delivery Systems, Marcel Dekker, Inc., New York, N.Y. (1993) and also Havener, W. H., Ocular Pharmacology, C.V. Mosby Co., St. Louis (1983)).

For oral administration, either solid or fluid unit dosage forms can be prepared. For preparing solid compositions such as tablets, the compound of interest is mixed into formulations with conventional ingredients such as talc, magnesium stearate, dicalcium phosphate, magnesium aluminum silicate, calcium sulfate, starch, lactose, acacia, methylcellulose, and functionally similar materials as pharmaceutical diluents or carriers.

Capsules are prepared by mixing the compound of interest with an inert pharmaceutical diluent and filling the mixture into a hard gelatin capsule of appropriate size. Soft gelatin capsules are prepared by machine encapsulation of a slurry of the compound of interest with an acceptable vegetable oil, light liquid petrolatum or other inert oil. Fluid unit dosage forms for oral administration such as syrups, elixirs and suspensions can be prepared. The water soluble forms can be dissolved in an aqueous vehicle together with sugar, aromatic flavoring agents and preservatives to form a syrup. An elixir is prepared by using a hydroalcoholic (e.g., ethanol) vehicle with suitable sweeteners such as sugar and saccharin, together with an aromatic flavoring agent. Suspensions can be prepared with an aqueous vehicle with the aid of a suspending agent such as acacia, tragacanth, methylcellulose and the like.

Appropriate formulations for parenteral use are apparent to the practitioner of ordinary skill, such as the use of suitable injectable solutions or suspensions. Usually, the compound of interest is prepared in an aqueous solution in a concentration of from about 1 to about 100 mg/ml. More typically, the concentration is from about 10 to 60 mg/ml or about 20 mg/ml. Concentrations below 1 mg/ml may be necessary in some cases depending on the solubility and potency of the compound selected for use. The formulation, which is sterile, is suitable for various topical or parenteral routes including intra-dermal, intramuscular, intravascular, and subcutaneous.

In addition to the compound of interest, the compositions may include, depending on the formulation and mode of delivery desired, pharmaceutically-acceptable, non-toxic carriers or diluents, which include vehicles commonly used to form pharmaceutical compositions for animal or human administration. The diluent is selected so as not to unduly affect the biological activity of the combination. Examples of such diluents which are especially useful for injectable formulations are water, the various saline, organic or inorganic salt solutions, Ringer's solution, dextrose solution, and Hank's solution. In addition, the pharmaceutical composition or formulation may include additives such as other carriers; adjuvants; or non-toxic, non-therapeutic, non-immunogenic stabilizers and the like.

Furthermore, excipients can be included in the formulation. Examples include cosolvents, surfactants, oils, humectants, emollients, preservatives, stabilizers and antioxidants. Any pharmacologically acceptable buffer may be used, e.g., tris or phosphate buffers. Effective amounts of diluents, additives, and excipients are those amounts which are effective to obtain a pharmaceutically acceptable formulation in terms of solubility, biological activity, etc.

The compound of interest maybe incorporated into a microsphere. The compound of interest can be loaded into albumin microspheres, form which it is possible to recover such microspheres in a dry powder for nasal administration. Other materials suitable for the preparation of microspheres include agar, alginate, chitosan, starch, hydroxyethyl starch, ovalbumin, agarose, dextran, hyaluronic acid, gelatin, collagen, and casein. The microspheres can be produced by various processes known to the person skilled in the art such as a spray drying process or an emulsification process.

For example, albumin microspheres can be prepared by adding rabbit serum albumin in phosphate buffer to olive oil with stirring to produce a water in oil emulsion. Glutaraldehyde solution is then added to the emulsion and the emulsion stirred to cross-link the albumin. The microspheres can then be isolated by centrifugation, the oil removed and the spheres washed, e.g., with petroleum ether followed by ethanol. Finally, the microspheres can be sieved and collected and dried by filtration.

Starch microspheres can be prepared by adding a warm aqueous starch solution, e.g., of potato starch, to a heated solution of polyethylene glycol in water with stirring to form an emulsion. When the two-phase system has formed (with the starch solution as the inner phase) the mixture is then cooled to room temperature under continued stirring whereupon the inner phase is converted into gel particles. These particles are then filtered off at room temperature and slurried in a solvent such as ethanol, after which the particles are again filtered off and laid to dry in air.

The microspheres can be hardened by well known cross-linking procedures such as heat treatment or by using chemical cross-linking agents. Suitable agents include dialdehydes, including glyoxal, malondialdehyde, succinicaldehyde, adipaldehyde, glutaraldehyde and phthalaldehyde, diketones such as butadione, epichlorohydrin, polyphosphate, and borate. Dialdehydes are used to cross-link proteins such as albumin by interaction with amino groups, and diketones form schiff bases with amino groups. Epichlorohydrin activates compounds with nucleophiles such as amino or hydroxyl to an epoxide derivative.

The term “unit dosage form” refers to physically discrete units suitable as unitary dosages for subjects, e.g., mammalian subjects, e.g., humans, dogs, cats, and rodents, each unit containing a predetermined quantity of active material calculated to produce the desired pharmaceutical effect in association with the required pharmaceutical diluent, carrier or vehicle. The specifications for the unit dosage forms of this invention are dictated by and dependent on (a) the unique characteristics of the active material and the particular effect to be achieved and (b) the limitations inherent in the art of compounding such an active material for use in humans and animals. Examples of unit dosage forms are tablets, capsules, pills, powder packets, wafers, suppositories, granules, cachets, teaspoonfuls, tablespoonfuls, dropperfuls, ampoules, vials, aerosols with metered discharges, segregated multiples of any of the foregoing, and other forms as herein described. The compositions can be included in kits, which can contain one or more unit dosage forms of the composition and instructions for use to treat one or more of the disorders described herein.

Slow or extended-release delivery systems, including any of a number of biopolymers (biological-based systems), systems employing liposomes, colloids, resins, and other polymeric delivery systems or compartmentalized reservoirs, can be utilized with the compositions described herein to provide a continuous or long term source of therapeutic compound. Such slow release systems are applicable to formulations for delivery via topical, intraocular, oral, and parenteral routes.

An effective quantity of the compound of interest is employed in treatment. The dosage of compounds used in accordance with the invention varies depending on the compound and the condition being treated. For example, the age, weight, and clinical condition of the recipient patient. Other factors include: the route of administration, the patient, the patient's medical history, the severity of the disease process, and the potency of the particular compound. The dose should be sufficient to ameliorate symptoms or signs of the disease treated without producing unacceptable toxicity to the patient. In general, an effective amount of the compound is that which provides either subjective relief of symptoms or an objectively identifiable improvement as noted by the clinician or other qualified observer.

Broadly, for a PPARγ activator, the oral dose can be determined from the following formula: oral dose (in mg)=(k₁)(EC₅₀)(k₂) (LBW) (MW), wherein k₁ is a constant of 5 to 100, EC₅₀ is the concentration (amount) of compound required to activate or bind to 50% of PPARγ in the sample or patient and is in mole/L units, k₂ is the fractional water content of the lean body weight (LBW) of the patient=0.72 L/kg (see Geigy Scientific Tables Vol. 1, Lentner (ed.), page 217, Giba-Geigy Ltd., Basle, Switzerland (1981)), and MW is the molecular weight of the compound in g/mole.

The compounds in this invention can also be given orally in combination with natural or synthetic compounds that bind to or modify the activity of the vitamin D receptor or in combination with compounds that bind to or modify the activity of the retinoid X receptor to provide for a synergistic effect in the treatment or prevention of the disorders. Examples of such compounds that provide for synergistic effect when given in combination with the drugs encompassed by the current invention include vitamin D analogs, various retinoic acid derivatives, and other activators for retinoid X receptors or retinoic acid receptors including but not limited to compounds such as LG100268, tazarotene, TTNPB, AGN 190121, adapalene, or LGD1069 (Targretin).

Each dosage unit for oral administration contains suitably from 0.1 mg to 500 mg/kg, e.g., from 0.1 mg to 50 mg/kg, e.g., from 0.2 mg to 20 mg/kg, and, e.g., from 0.2 mg to 2 mg/kg, and each dosage unit for parenteral administration can contain from 0.1 mg to 100 mg/kg of a compound of the cannabinoid or derivative thereof calculated as the free base. Each dosage unit for intranasal administration can contain 1-400 mg, e.g., 10 to 200 mg per person.

V. Uses of Cannabinoids as PPAR Activators

In one aspect, the invention pertains to using cannabinoids, e.g., AJA for modifications of disorders associated with PPAR, e.g., PPARγ. The present invention relates to compounds, pharmaceutical compositions containing them, methods for preparing the compounds, and their use as medicaments. More specifically, compounds of the invention can be utilized in the treatment of conditions mediated by nuclear receptors, in particular the PPAR. The present compounds reduce blood glucose and triglyceride levels and are accordingly useful for the treatment of ailments and disorders such as diabetes and obesity.

(i) Use of AJA in Diabetes

In one embodiment, the invention pertains to using AJA as an activator of PPARγ as a method for modifying an autoimmune disorder such as diabetes. Diabetes is a multifactorial disease that occurs through the failure and/or destruction of the pancreatic β-cell. There is a large body of evidence in support of the idea that inflammatory cytokines have cytotoxic effects on islet β-cells (Rabinovitch, J Clin Endocrinol Metab. 71:152-6, 1990) and this cytotoxicity plays a part in β-cell destruction in insulin-dependent diabetes mellitus (IDDM).

Obesity-linked non-insulin-dependent diabetes mellitus (NIDDM) is preceded by years of insulin resistance, during which normal blood glucose levels are maintained through effective compensation by pancreatic β-cells. In approximately 20% of obese individuals, the compensation wanes, hyperglycemia appears, and overt NIDDM is diagnosed. The depressed β-cell function is thought be due to excess free fatty acids released from adipocytes in obesity (Campbell et al., Am J Physiol. 266: E600-5., 1994; DeFronzo, Diabetes Metab Rev. 4:727-47, 1988) acting to initially stimulate, but ultimately impair, the function of β-cells, and thus limit their compensatory capability. Thus, impaired β-cell function is a characteristic of both IDDM and NIDDM.

The compounds are useful for the modification, treatment, and/or prophylaxis of insulin resistance (Type II diabetes), impaired glucose tolerance, dyslipidemia, disorders related to Syndrome X such as hypertension, obesity, insulin resistance, hyperglycemia, atherosclerosis, hyperlipidemia, coronary artery disease and other cardiovascular disorders associated with diabetes. The disorders also include those associated with diabetes, e.g., the anti-apoptotic effect of PPARγ activators that serves to protect cells from premature death and promote their survival, as in degenerative and dystrophic diseases, e.g., retinal neural and glial cells in diabetic retinopathy and both “wet” (exudative) and “dry” (aereolar) age-related macular degeneration.

(ii) Use of AJA in Lipid Metabolism and Glucose Homeostasis

In another embodiment, the invention pertains to using AJA as an activator of PPARγ, for the treatment or modification of disorders related to lipid metabolism and energy homeostasis. Also, compounds that block PPARγ activity would be useful for interfering with the maturation of preadipocytes into adipocytes and thus would be useful for the treatment of obesity and related disorders associated with undesirable adipocyte maturation.

Adipose tissue plays a central role in lipid homeostasis and the maintenance of energy balance in vertebrates. Adipocytes store energy in the form of triglycerides during periods of nutritional affluence and release it in the form of free fatty acids at times of nutritional deprivation. The development of white adipose tissue is the result of a continuous differentiation process throughout life. Much evidence points to the central role of PPARγ activation in initiating and regulating this cell differentiation. Several highly specialized proteins are induced during adipocyte differentiation, most of them being involved in lipid storage and metabolism. The exact link from activation of PPARγ to changes in glucose metabolism, most notably a decrease in insulin resistance in muscle, has not yet been clarified. A possible link is via free fatty acids such that activation of PPARγ induces Lipoprotein Lipase (LPL), Fatty Acid Transport Protein (FATP) and Acyl-CoA Synthetase (ACS) in adipose tissue but not in muscle tissue. This, in turn, reduces the concentration of free fatty acids in plasma dramatically, and due to substrate competition at the cellular level, skeletal muscle and other tissues with high metabolic rates eventually switch from fatty acid oxidation to glucose oxidation with decreased insulin resistance as a consequence. PPARγ is involved in stimulating β-oxidation of fatty acids.

(iii) Use of AJA in Inflammation Involving PPARγ

In another embodiment, the invention pertains to using AJA as an activator of PPARγ, for the treatment or modification of disorders related to those inflammatory responses involving PPARγ activation. The cannabinoid compounds, e.g., AJA, can be used for the treatment of diseases including, but not limited to, immunologically-mediated inflammatory diseases such as rheumatoid arthritis, systemic lupus erythematosus, psoriasis, eczema, multiple sclerosis, diabetes and thyroiditis. In addition, the present compounds can modulate bone formation/resorption and are useful in the treatment of conditions including but not limited to ankylosing spondylitis, gout, arthritis associated with gout, osteoarthritis and osteoporosis.

The invention will be further described in the following examples, which do not limit the scope of the invention described in the claims.

### EXAMPLES

### Example 1

### Materials and Methods

(i) Reagents

The PPARγ-specific ligand, GW347845, was obtained from GlaxoSmithKline. The 9-cis retinoid acid (9c-RA) was obtained from Activator Pharmaceuticals. Ajulemic acid was obtained from Organix Inc (Woburn, Mass.). All other chemicals were purchased from commercial sources (e.g., Sigma).

(ii) Plasmids

The pGEX-DRIP205 (527˜970) was obtained from the Sloan-Kettering Cancer Center. The Gal4-hPPAR LBD constructs were obtained from GlaxoSmithKline, Research Triangle Park, NC. The IL8-Luciferase reporter was obtained from Celgene, San Diego, Calif. The plasmids mPPARα, mPPARγ1, mPPARδ , and hRXRα were in the pCMX vector (Umesono et al., Cell 65:1255-1266, 1991).

The-pCMX-Gal4-mPPARγ1 was constructed by fusing the mPPARγ1 coding sequence to the yeast GAL4 DNA binding domain (amino acid 1-147) in the pCMX vector. The AF-2 helix-truncated mutant of mPPARγ1 (PPARγ ΔAF2) was generated by PCR amplification-to introduce a stop codon and a NheI restriction site after amino acid 489, and then-subcloned into the pCMX vector. The PPRE-TK-LUC, MH100-TK-LUC-and DR1-TK-LUC reporters have been described previously (Forman et al., Cell 83:803-812, 1995; Umesono et al., Cell 65:1255-1266, 1991).

(iii) Cell Culture and Transient Transfection

For PPARγ activation assay, HEK293 cells were plated in 12-well cell culture plates and maintained in Dulbecco's modified Eagle's medium (DMEM) supplemented with 10% fetal bovine serum (FBS) at 37° C. under 5% CO₂. Cells were changed to phenol red-free DMEM supplemented with 10% charcoal-stripped fetal bovine serum 3 hours before transfection by a standard calcium-phosphate precipitation method. Twelve hours after transfection, the cells were washed with phosphate-buffered saline (PBS) and fed again with fresh medium containing the indicated concentrations of compounds. After 36 hours, cells were harvested for β-galactosidase and luciferase activities as described previously (Li et al., Proc Natl Acad Sci USA 94:8479-84, 1997). The average normalized luciferase activity was determined via triplicate experiments. For IL-8 promoter assay, HeLa cells were maintained and transfected in the same way as described above. After transfection, cells were recovered for 4 hours before treatment with AJA, GW347845, or solvent. After 3-4 hours, cells were treated with or without 25 nM of phorbol myristate acetate (PMA) for 24 hours.

(iv) Partial Protease Digestion Assay

Partial protease digestion was carried out as described by Leng et. al. (J Steroid Biochem Mol Biol 46:643-61, 1993). PPAR proteins were made by in vitro transcription/translation reactions in reticulocyte lysate according to the manufacture's instructions (Promega). AJA, GW347845, or vehicle alone was incubated with the 35S-labeled PPARs at room temperature for one hour before trypsin digestion. Reactions were stopped by boiling in SDS-containing sample buffer, and lysates were subjected to SDS-PAGE and autoradiography.

(v) Expression and Purification of GST Fusion Protein

The glutathione S-transferase (GST)-DRIP205 (amino acid 527-970) and GST-RAC3 RID (amino acid 613-752) fusion proteins were expressed in BL21 cells by induction with 0.1 mM isopropyl-β-D-thiogalactopyranoside (IPTG) at room temperature for 4 hours. The bacterial pellet was resuspended in STE buffer (150 mM NaCl, 10 mM Tris pH 8.0, 1 mM EDTA), containing 5 mM dithiothreitol (DTT), 1 mM phenylmethlsulfonyl fluoride (PMSF) and 1.5% Sarkosyl. The cell suspension was sonicated and centrifuged at 7400 rpm at 4° C. for 30 minutes. The supernatant was isolated and 0.02% Triton-X 100 was added, and the mixture was then incubated with 1 ml 50% slurry of glutathione-Sepharose beads on a rotator at 4° C. for 30 minutes. The beads were spun down at 3000 rpm for 10 minutes, the supernatant was removed, and the beads were suspended in 1 ml cold PBS.

(vi) GST Pull-Down Assay

Approximately 5 μg of GST-DRIP205 or GST-RAC3 bound on glutathione-Sepharose beads and 4 μl ³⁵S-methionine-labeled mPPARγ1 were incubated with the indicated concentrations of AJA, GW347845, or vehicle alone in H buffer (20 mM Hepes [pH 7.7], 75 mM KCl, 0.1 mM EDTA, 2.5 mM MgCl₂, 0.05% NP40, 0.1 mM methionine, 1 mM DTT) containing 1 mg/ml bovine serum albumin (BSA) on a rotator at 4° C. overnight. After three washes with cold PBS, the bound protein was eluted in SDS sample buffer and boiled for 10 min before SDS-PAGE and autoradiography. To ensure that equal amounts of GST fusion proteins were recovered in the pull-down assay, the gel was stained with Coomassie blue before autoradiography.

(vii) Adipocyte Differentiation Assay and RT-PCR

The adipocyte differentiation assay was performed as described by Mukherjee et. al. (Mol Endocrinol 14: 1425-33, 2000). The 3T3 L1 cells (American Type Culture Collection) were cultured in DMEM media supplemented with 10% calf serum. Two days after reaching confluence, cells were treated with AJA, GW347845, or vehicle (0.1% DMSO) in the presence of 10 μg/ml insulin every other day. After 10 days of treatment with AJA or 7 days with GW347845 at confluence, cells were fixed and stained with Oil Red O (Sigma). For RT-PCR, total cellular RNAs were isolated by TRIzol (Gibco). Reverse transcriptase polymerase chain reaction (RT-PCR) was performed using the superscript first-strand synthesis kit (Invitrogen). After first-strand cDNA was synthesized by use of oligo (dT), cDNA was amplified by PCR. The forward primers and reverse primers used in the amplifications were 5′-GCT GTT ATG GGT GAA ACT CTG GGA G-3′(SEQ ID NO: 1), and 5′-CTT CAT GAG GCC TGT TGT AGA GC-3′ (SEQ ID NO:2), for PPARγ2, 5′-GAG CAA ATG GAG TTC CCA GAT G-3′ (SEQ ID NO: 3), and 5′-GCA AAC AAT GGG AAT AGT TCA CAG TAG-3′ (SEQ ID NO: 4), for aP2, and 5′-GAC CAC AGT CCA TGC CAT CAC-3′(SEQ ID NO: 5), and 5′-CAT ACC AGG AAA TGA GCT GAC-3′ (SEQ ID NO: 6), for GAPDH. PCR amplifications were performed in 50 μL volume with Taq DNA polymerase for 30 cycles.

### Example 2

### Binding of Ajulemic Acid to PPARγ

To determine if AJA can bind to PPARγ, in vitro translated ³⁵S-labeled mPPARγ1 was incubated with AJA or a carrier vehicle before trypsin digestion. To test if AJA binds to PPARs, partial proteinase digestion assays were conducted as described previously (Leng et al., J Steroid Biochem Mol Biol 46:643-61, 1993). Ligand binding is known to cause conformational changes of nuclear receptors, which is an essential process for transcriptional activation by the receptors (Allen et al., J Biol Chem 267:19513-20, 1992). Such conformational change can be detected by limited proteinase digestion based on alteration of the accessibility of proteolytic sites on the surface of the receptors.

The PPAR proteins were synthesized and labeled with ³⁵S-methionine in reticulocyte lysate and incubated with AJA, GW347845, or vehicle alone at room temperature for one hour before trypsin digestion. The reactions were terminated by boiling in SDS-containing protein sample buffer. The proteolytic patterns of PPAR proteins were then analyzed by SDS-PAGE and autoradiography, and compared with vehicle-treated control samples.

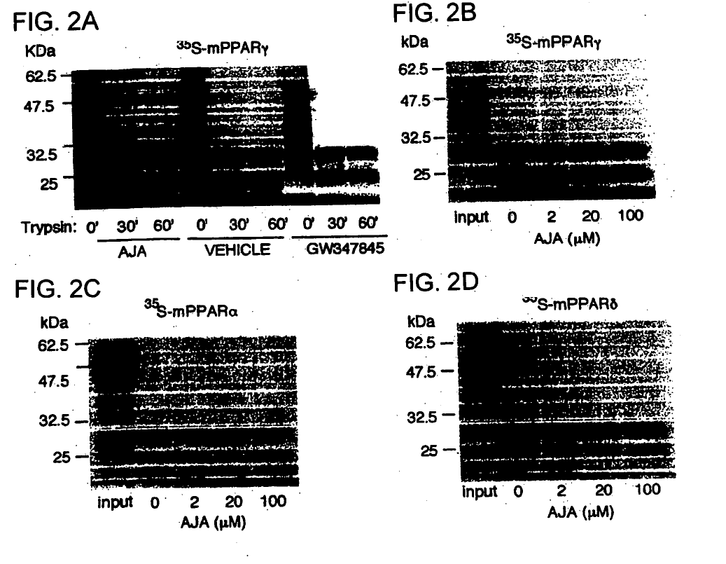

In the experiment illustrated in FIG. 2A, the final concentrations of ligands were 20 μM AJA and 1 μM GW347845 in 0.2% DMSO (vehicle). Partial proteinase digestions were conducted with 30 μg/ml of trypsin for 30 or 60 minutes as indicated. FIG. 2A shows a clear difference in the proteolytic profiles between AJA and vehicle-treated PPARγ proteins was observed. Two prominent trypsin-resistant fragments of 30 and 24 kDa, respectively, were detected after incubation with 20 μM AJA when compared with the control sample, in which the 30-kDa band disappeared completely after 60 min of digestion and two smaller fragments emerged after 30 min of digestion. As a positive control, the potent PPARγ agonist GW347845 (Cobb et al., J Med Chem; 41:5055-5069, 1998; Suh et al., Cancer Res 59:5671-3, 1999), also produced a proteinase digestion pattern similar to the one seen in AJA-treated sample. These findings show that AJA treatment can cause a conformational change in PPARγ, demonstrating that AJA may bind directly to PPARγ.

The AJA concentrations required to protect PPARγ from trypsin digestion were further measured. The two proteinase-resistant PPARγ fragments were observed at 2, 20 and 100 μM AJA concentrations, with a slight dose-dependent increase between 2 and 20 μM (FIG. 2B). In contrast, AJA at all of these concentrations did not affect the proteinase sensitivity of PPARα (FIG. 2C) or PPARδ (FIG. 2D). These data indicate that AJA binds specifically to PPARγ, but it does not interact with PPARα or PPARδ, suggesting that AJA is a selective ligand for PPARγ.

### Example 3

### Ajulemic Acid Activates PPARγ

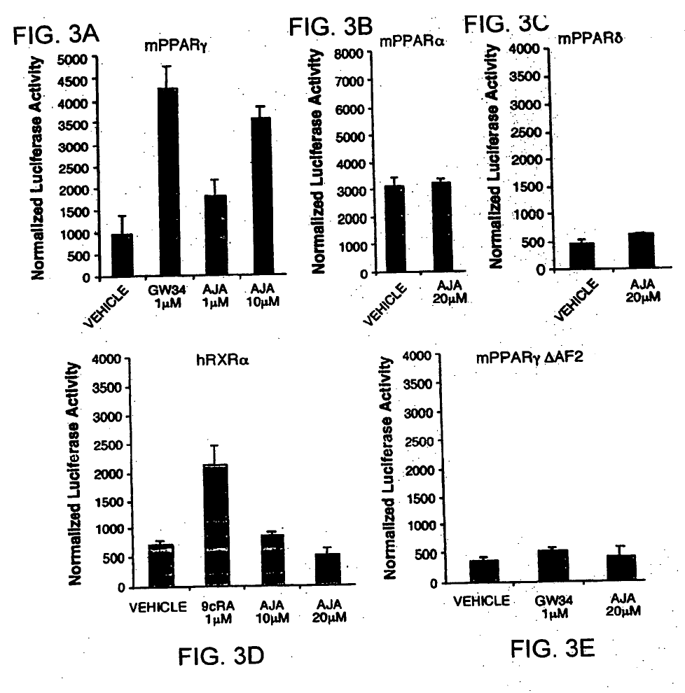

The observation that AJA induces conformational change of PPARγ prompted the investigation of whether AJA was a PPARγ agonist, and could activate PPARγ transcriptional activity. Therefore, transient transfections were performed to investigate this possibility. The PPRE-TK-LUC reporter (Kliewer et al., Proc Natl Acad Sci USA 91:7355-9, 1994) was cotransfected with the PPARγ expression vector into the human kidney HEK293T cells followed by AJA treatment of the cells. The average luciferase activities were normalized with the cotransfected β-galactosidase in triplicate experiments. After transfections, cells were treated with indicated concentrations of ligands or vehicle alone for 36 hours before harvesting for β-galactosidase and luciferase assays. In this functional assay, 1 μM GW347845 stimulated the transcriptional activity of PPARγ 4 fold. AJA also stimulated PPARγ transcriptional activity 2 to 3 fold at 1 to 10 μM concentrations in a dose-dependent manner (FIG. 3A). These results suggest that AJA indeed can activate the transcriptional activity of PPARγ, consistent with its ability to bind PPARγ and induce an active conformation.

In an effort to confirm that AJA activates PPARγ selectively, the ability of AJA to activate PPARα and PPARδ was analyzed in a similar transient transfection assay. PPARα had a 3-fold higher basal transcriptional activity than PPARγ in the absence of ligand; however, no further activation was observed after treatment with 1 to 20 μM AJA concentrations (FIG. 3B and data not shown). Similarly, neither did AJA activate PPARδ in this assay (FIG. 3C). These data indicate that AJA activates PPARγ selectively, consistent with its ligand-binding specificity.

PPARγ forms a permissive heterodimeric complex with the retinoid X receptor (RXR) and ligands for either PPARγ or RXR can both activate the receptor heterodimer (for a review, see reference Leblanc et al., Genes Dev 9:1811-6, 1995). In order to rule out the possibility that AJA might act through RXR, the ability of AJA to activate RXRα on a DR1-LUC reporter was analyzed, where RXR is known to form a homodimer that can be activated only by RXR-specific ligands. The hRXRα and a DR1-TK-LUC reporter were cotransfected into HEK293 cells, followed by 9-cis RA or AJA treatments. In this experiment, it was found that the RXR-specific ligand 9-cis RA activated the reporter gene expression 3 fold, but AJA failed to activate the reporter at saturating concentrations (FIG. 3D), suggesting that AJA does not bind or activate RXRα. These data indicate that the transcriptional activation of PPARγ by AJA in vivo is not mediated through its heterodimeric partner RXRα.

PPARγ contains two transcriptional activation domains: a constitutive N-terminal AF-1 domain and a ligand-dependent C-terminal AF-2 domain. The AF-2 function depends on the presence of an AF-2 helix (helix 12) located at the extreme C-terminal end of LBD. To examine whether activation of PPARγ by AJA is mediated through the ligand-dependent AF-2 function, the AF-2 helix of PPARγ was deleted to create a PPARγ ΔAF2 mutant, and then tested as to whether AJA could still activate the PPRE-Luc reporter through the ΔAF2 mutant. AJA and GW347845 both failed to activate expression of the reporter gene (FIG. 3E), suggesting that activation of PPARγ by AJA is mediated specifically through the AF-2 function. This is also consistent with the idea that AJA is an activating ligand for PPARγ.

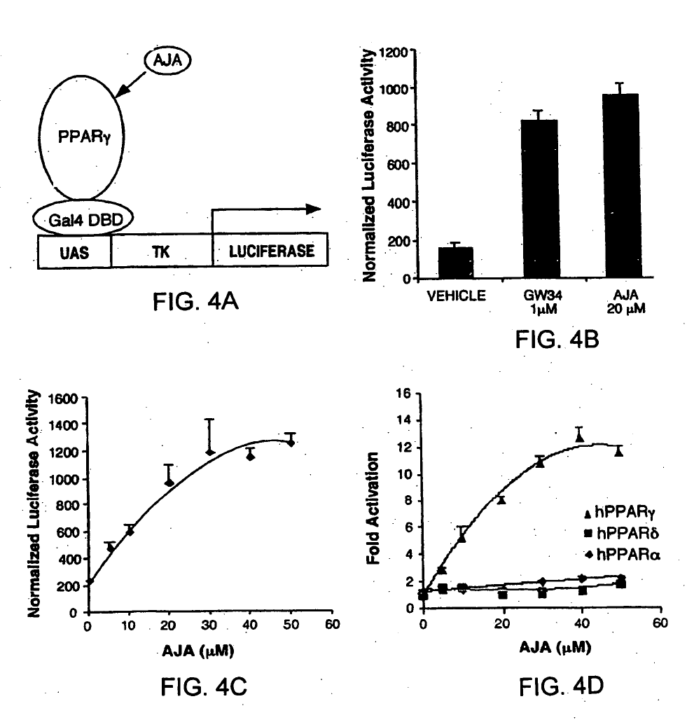

To further confirm activation of PPARγ by AJA, and also to determine PPARγ species specificity, the ability of AJA to activate the chimeric Gal4-DBD/PPAR fusion proteins on a Gal4-dependent MH100-Luc reporter was assessed (FIG. 4A). The PPARs were expressed as Gal4 DBD fusion proteins, which bind to the UAS promoter containing 4 copies of a synthetic Gal4 binding site upstream of the minimal thymidine kinase (TK) promoter. Transient transfection was conducted in HEK293 cells with the mPPARγ expression vector and the MH100-tk-Luc reporter. The Gal4 DBD/PPAR fusion protein will activate reporter gene expression in response to agonist binding to PPAR. In this system, activation of the reporter will be mediated exclusively through the chimeric Gal4-DBD fusion protein, thus eliminating potential interference from endogenous receptors. In this assay, both AJA and GW347845 activated the reporter gene expression significantly (FIG. 4B). Transfected cells were treated with increasing concentrations of AJA from 10-60 μM and the luciferase activities were determined in triplicate experiments. The estimated EC-50 for activation of PPARγ by AJA is 13 μM in this assay. The activation of Gal4-DBD/mPPARγ by AJA was concentration-dependent, with an estimated EC-50 of approximately 13 μM (FIG. 4C). The efficacy of AJA to activate human PPARγ in this assay was also tested, and confirmed that AJA also activates human PPARγ as efficiently as mouse PPARγ (FIG. 4D). Consistently, AJA did not activate human PPARα or PPARδ. Taken together, these data strongly suggest that AJA activates both mouse and human PPARγ specifically at pharmacologically relevant concentrations.

### Example 4

### AJA Stimulates Coactivator Binding to PPARγ

This example demonstrates that AJA-bound PPARγ can recruit coactivators. Nuclear receptor coactivators are known to interact with ligand-activated receptors to enhance transcriptional activation by recruiting chromatin modifying enzymes and RNA polymerase. The receptor-associated coactivator-3 (RAC3) of the p160/SRC family, and the DRIP205 subunit of the DRIP coactivator complex are known PPARγ coactivators (Yang et al., Mol Cell Biol 20:8008-17, 2000).

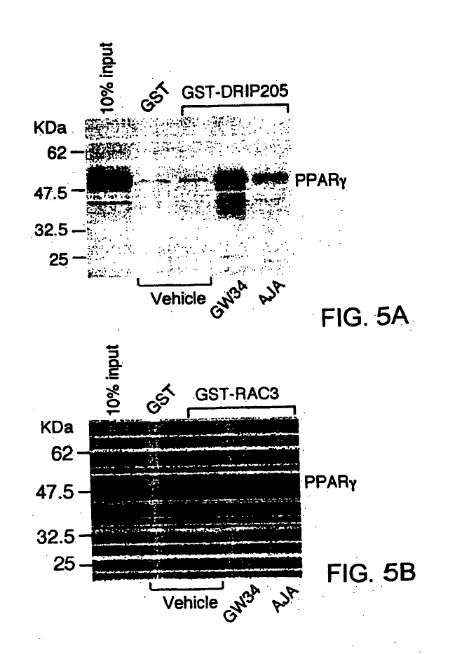

To evaluate further the mechanism whereby AJA influences PPARγ transcriptional activity, the ability of PPARγ to interact with DRIP205 and/or RAC3 in response to AJA treatment by GST-pull down assay was examined. The GST-DRIP205 (amino acids 527-970) and the GST-RAC3 (amino acids 613-752) fusion proteins were purified and used in binding reactions containing in vitro translated ³⁵S-labeled PPARγ in the presence of vehicle (0.1% DMSO), GW347845 (GW34, 1 μM), or AJA (20 μM). GST alone was used as a negative control in the pull down reaction. In this experiment, the 35S-methionine-labeled PPARγ showed negligible binding to GST alone, GST-DRIP205 or GST-RAC3 in the absence of ligand (FIGS. 5A and 5B). In contrast, AJA and GW347845 pretreatment of PPARγ caused increased binding of PPARγ to the GST-DRIP205 and GST-RAC3. The interaction between PPARγ and DRIP205 appeared to be stronger than the interaction between PPARγ and RAC3, consistent with the fact that DRIP205 is a more potent coactivator than RAC3 for PPARγ (Yang et al., Mol Cell Biol 20:8008-17, 2000; Zhu et al., J Biol Chem 272:25500-6, 1997). These data indicate that AJA treatment can promote the interaction of PPARγ with nuclear receptor coactivators, corroborating further the hypothesis that AJA binds directly to PPARγ and induces a transcriptionally active conformation of the receptor.

### Example 5

### Reduction of IL-8 Promoter Activity by Ajulemic Acid Through PPARγ

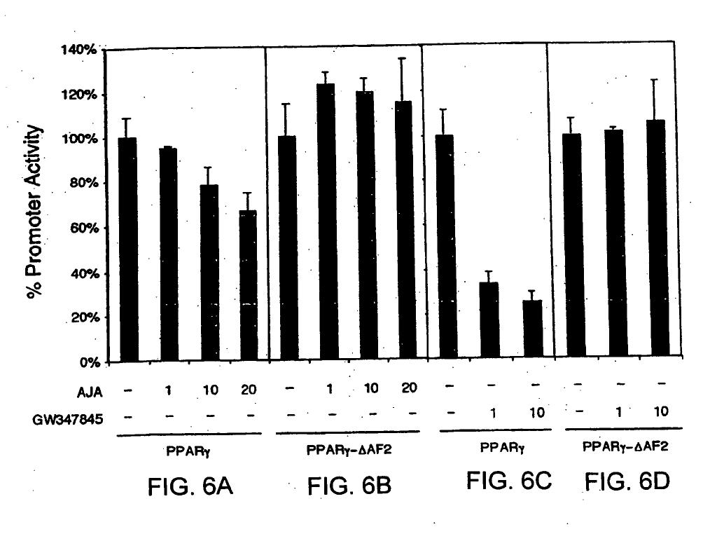

This example demonstrates the link between activation of PPARγ by AJA to the antiinflammatory activity of AJA. The IL-8 luciferase reporter controlled by the human IL-8 promoter (-97/-69) was cotransfected with mPPARγ or mPPARγ ΔAF2 mutant into HeLa cells. After transfection, cells were treated with AJA, GW347845 or solvent after PMA treatment for 24 hours. This was performed by determining the effect of AJA on IL-8 promoter activity and the role of PPARγ in this process. IL-8 is a biomarker for inflammation, and reduction of IL-8 levels correlates with a decrease in inflammation. The involvement of PPARγ in regulating IL-8 promoter activity was determined by comparing the IL-8 promoter activity in the presence of wild type PPARγ or its ΔAF2 mutant. In the experiment illustrated in FIG. 6A, PMA stimulated IL-8 promoter activity 3 fold, which was set as 100% promoter activity. AJA treatment reduced the IL-8 promoter activity by about 40% at 20 μM concentration in cells transfected with the wild type PPARγ. This effect appears concentration-dependent, and the reduction of IL-8 promoter activity is statistically significant at 10 and 20 μM AJA concentrations. AJA had no effect on IL-8 promoter activity in cells transfected with the PPARγ ΔAF2 mutant (FIG. 6B), suggesting that the reduction of IL-8 promoter activity is mediated through activation of PPARγ by AJA. As a control, GW347845 dramatically reduced IL-8 promoter activity in cells cotransfected with the wild type PPARγ, but not with the ΔAF2 mutant (FIGS. 6C and D). These data indicate that activation of PPARγ by AJA is involved in the inhibition of IL-8 promoter activity, suggesting a putative mechanism for the antiinflammatory action of AJA.

### Example 6

### Induction of Adipocyte Differentiation by Ajulemic Acid

To provide biological evidence that AJA is a bona fide activator for PPARγ, the ability of AJA to induce differentiation of 3T3 L1 fibroblasts into adipocytes was assessed. Activation of PPARγ by its ligands is an essential process for the onset of adipocyte differentiation, which is characterized by morphological changes, droplet formation, and induction of adipocyte-specific genes such as PPARγ2 and aP2 (Barak et al., Mol Cell 4:585-95, 1999; Rosen et al., Genes Dev 16:22-6, 2002; Tontonoz et al., Cell 79:1147-56, 1994). PPARγ ligands can substitute for the adipogenic hormones during differentiation of preadipocytes into adipocytes (Chawla et al., Proc Natl Acad Sci USA 91:1786-90, 1994), and ectopically expressed PPARγ is able to transdifferentiate myoblasts into adipocytes (Hu et al., Proc Natl Acad Sci USA 92:9856-60, 1995).

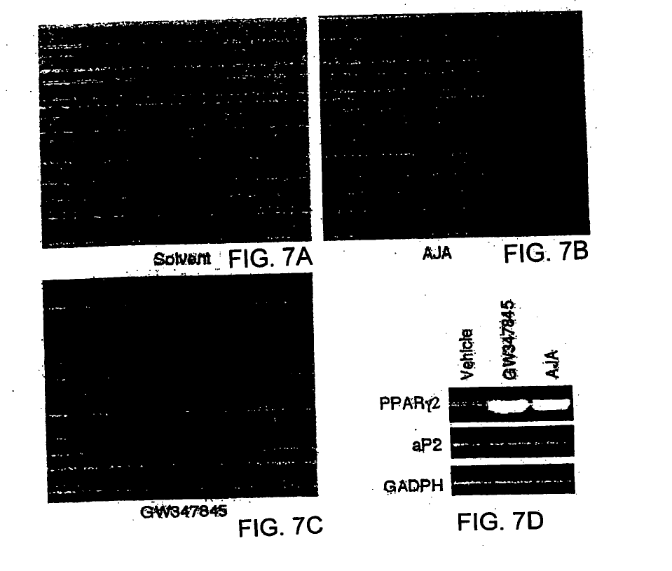

3T3 L1 fibroblasts cells were cultured in DMEM supplemented with 10% calf serum. Two days after confluence, cells were treated with 0.1% DMSO, 20 μM AJA, or 1 μM GW347845 in the presence of 10 μg/ml of insulin. The cells were treated for 10 days with AJA, or 7 days with GW347845, and lipid accumulation in cells was assessed by Oil Red O staining. A dramatic increase in lipid droplet staining in the cytoplasm was observed after treatment with AJA or GW347845 (FIG. 7A), suggesting that both AJA and GW347845 can induce differentiation of 3T3 L1 fibroblasts into adipocytes.

To confirm that AJA and GW347845 induce 3T3 L1 cell differentiation into adipocytes, the expression levels of the adipocyte-specific genes PPARγ2 and aP2 were measured by RT-PCR. Total RNA was isolated after AJA or GW347845 treatment, and compared with vehicle control. Reverse transcription was conducted and the relative amounts of PPARγ2 and aP2 transcripts were measured by PCR reactions using primer sets specific to each gene. PCR products were analyzed on 1% agarose gel and stained with Ethidium Bromide. Both AJA and GW347845 enhanced PPARγ2 and aP2 gene expression significantly in comparison with vehicle control treatment (FIG. 7B). As a control for the induction specificity and the PCR amplification reaction, the expression levels of GADPH were measured in all samples, and found not affected by any treatment. These data indicate that AJA can induce differentiation of 3T3 L1 fibroblasts into adipocytes, further suggesting that AJA is an active ligand for PPARγ.

The molecular basis for the therapeutic action of ajulemic acid (AJA), were investigated. The data upon which the invention is based, in part, demonstrate that AJA can bind selectively to PPARγ in vitro and that AJA activates the transcriptional activity of PPARγ in vivo. Activation of PPARγ by AJA depends on the presence of the AF-2 helix in the receptor. AJA binding enables PPARγ to recruit nuclear receptor coactivators. In addition, AJA inhibits IL-8 promoter activity in a PPARγ-dependent manner, and induces differentiation of 3T3 L1 fibroblasts into adipocytes. The data suggest that AJA exerts its therapeutic actions, at least in part, through activation of PPARγ.

Based on the structural and functional similarity of AJA and several known PPARγ ligands, it was demonstrated that AJA binds to PPARγ. In a partial proteinase digestion assay, AJA effectively protects PPARγ from proteinase digestion (FIGS. 2A-D), reflecting direct binding of AJA to PPARγ. The two trypsin resistant fragments likely contain the ligand-binding domain (LBD) of PPARγ, because ligand binding induces a compact conformation in the LBD (Xu et al., Proc Natl Acad Sci USA 98:13919-24, 2001), which is expected to be more resistant to proteinase digestion when compared with the unliganded receptor. Because AJA causes proteinase resistance only to PPARγ, but not PPARα or PPARδ, it is clear that AJA binds selectively to PPARγ. Indeed, the three PPARs have distinct ligand binding specificities (Xu et al., Proc Natl Acad Sci USA 98:13919-24, 2001), and physiological functions (Berger et al., Annu Rev Med 53:409-35, 2002). The selective binding of AJA to PPARγ suggests that PPARγ may mediate the therapeutic activity of AJA.

Consistent with the binding of AJA to PPARγ, the transient reporter gene assay further demonstrates that AJA activates the target promoter through PPARγ, but not PPARα or PPARδ (FIGS. 3A-E). These data indicate that AJA is a PPARγ-specific agonist, a conclusion that is further supported by the inability of AJA to activate the PPARγ ΔAF2 mutant, which is defective in ligand-dependent transcriptional activation (FIG. 3E). The observation that PPARγ activation by AJA requires the AF-2 function is consistent with the hypothesis that AJA binds to PPARγ LBD and activates its ligand-dependent transcriptional function. By using the Gal4-DBD fusion protein system, the EC-50 of AJA for PPARγ activation was measured to be 13 μM (FIGS. 4A-D), a concentration that is within the pharmacologically effective doses of AJA (Burstein, Curr Pharm Des 6:1339-45, 2000). Furthermore, AJA also activates human PPARγ equally well compared with mouse PPARγ, implying that the PPARγ-dependent therapeutic activity of AJA observed in mice might be applicable to humans.

As an agonist for PPARγ, AJA's binding enables PPARγ to recruit nuclear receptor coactivators (FIGS. 5A and 5B). Nuclear receptor coactivators are known to interact with liganded receptors to facilitate transcriptional activation of a target promoter by recruiting histone acetyltransferase activity to the receptor (Leo and Chen 2000). The interaction of PPARγ with DRIP205 in response to AJA treatment appears to be more prominent than the interaction with RAC3. This is consistent with a report showing that DRIP205 is a potent coactivator for PPARγ (Yang et al., Mol Cell Biol 20:8008-17, 2000). AJA may induce formation of a coactivator-binding surface to allow docking of the coactivator LXXLL motif. Furthermore, addition of AJA causes differentiation of 3T3 L1 fibroblasts into adipocytes (FIGS. 7A-D), a process that is known to be mediated by PPARγ. Together, these data provide strong evidence that AJA is an agonist of PPARγ. These data also demonstrate the involvement of PPARγ in the signaling of this cannabinoid class of analgesic and antiinflammatory drugs.

Activation of PPARγ is, at least partly, responsible for the antiinflammatory action of AJA, and other cannabinoids as well. In fact, several natural cannabinoids, such as THC and cannabidiol (CBD), also activate PPARγ. The involvement of PPARγ in AJA-mediated antiinflammatory activity is further supported by the observation that AJA inhibits IL-8 promoter activity in a PPARγ-dependent manner (FIGS. 6A-D). This inhibition occurs only in the presence of wild type PPARγ, but not the ΔAF-2 mutant, suggesting that transcriptional activation by PPARγ is required for the repression of IL-8 promoter activity by AJA. It is not clear whether inhibition of cytokine promoter activity by PPARγ is due to direct binding of PPARγ to the promoter. Since there is no evidence for direct binding of PPARγ to the cytokine promoter, it is possible that this inhibition is indirect.

Treatment of peritoneal macrophages with several PPARγ ligands such as 15d-PGJ₂ also suppresses expression of the inducible nitric oxide synthase (iNOS), gelatinase B, and scavenger receptor A in response to phorbol ester stimulation. The promoters of these genes were found to possess binding sites for activator protein-1 (AP-1), nuclear factor κB (NF-κB), and the signal transducer and activator of transcription (STAT). Furthermore, the inhibition of inflammatory response by PPARγ ligands in macrophages was produced in part by antagonizing the activities of these transcription factors (Ricote et al., Nature 391:79-82, 1998). Although 15d-PGJ₂ was shown in some studies to be able to mediate an anti-inflammatory action in a PPARγ-independent manner (Straus et al., Proc Natl Acad Sci USA 97:4844-9, 2000; Tsubouchi et al., Biochem Biophys Res Commun 283:750-5, 2001), the inhibition of inflammatory responses by activation of PPARγ was confirmed by several recent studies both in vitro (Ji et al., J Autoimmun 17:215-21, 2001) and in vivo (Dubuquoy et al., Gastroenterol Clin Biol 24:719-24, 2000; Kawahito et al., J Clin Invest 106:189-97, 2000; Naito et al., Aliment Pharmacol Ther 15:865-73, 2001).

It is interesting to note that, like the activation of PPARγ, the immunosuppressive functions of some cannabinoids were also reported to be exerted by the inhibition of NF-κB, STAT, and AP-1 (Faubert et al., J Leukoc Biol 67:259-66, 2000; Jeon et al., Mol Pharmacol 50:334-41, 1996; Zheng et al., Biochem Pharmacol 51:967-73, 1996). The complete mechanism of the antiinflammatory action of AJA is likely to involve a complex signaling network. PPARγ is highly expressed in several immune cells, such as macrophages and monocytes. Therefore, it is possible that AJA may activate PPARγ in these cells to modulate immune inflammatory responses. Because PPARγ is involved in several physiological processes including lipid metabolism, glucose homeostasis, and adipocyte differentiation, the discovery that AJA is an active ligand for PPARγ led to the discovery that AJA may has a broader range of pharmacological activities than previously expected. For example, AJA is a useful agent for treatment of diabetes mellitus.

### Example 7

### Treatment of a Patient with Diabetes Mellitus

A 53 year old male patient who weighs 105 kg is diagnosed by his physician as having diabetes mellitus. The patient is mildly obese, has high blood sugar, and exhibits early signs of atherosclerosis. The physician prescribes treatment with ajulemic acid.

Accordingly, under physician supervision the patient takes an oral dose of 10 mg of ajulemic acid (in a pharmaceutically and palatably acceptable carrier) once every two weeks for three months. The physician expects this course of treatment to cause the patient to lose weight at a healthy rate, lower the patient's blood sugar back into the normal healthy range, and slow progression of the patient's atherosclerosis.

### Example 8

### Treatment of a Patient with Rheumatoid Arthritis

A 62 year old female patient who weighs 70 kg is diagnosed by her rheumatologist as having severe rheumatoid arthritis. The patient feels stiffness and prolonged soreness and aching in her joints, particularly in her elbow, knee, and shoulder joints, which has limited her movement and mobility. She feels general fatigue. Her rheumatoid factor is also consistently measured to be outside the normal range. The rheumatologist prescribes treatment with ajulemic acid.

Once per month the patient visits her rheumatologist for an injection into her arm of 15 mg of ajulemic acid (delivered in a pharmaceutically acceptable carrier). This course of treatment continues indefinitely. The rheumatologist expects treatment with ajulemic acid to reduce inflammation in the patient's joints, thus alleviating the stiffness, soreness, and aching, and allowing enhanced mobility.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject having a disorder associated with peroxisome proliferator-activated receptor-gamma (PPARγ) function, the method comprising administering to the subject a pharmaceutically effective amount of a composition comprising a cannabinoid compound.

2. The method of claim 1, wherein the cannabinoid has the general formula: 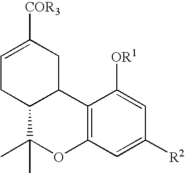
wherein R¹ is a hydrogen atom, —COCH₃ or —COCH₂CH₃, R² is a branched C₅-C₁₂ alkyl, and R³ is OH, OCH₃, or NHCH₂COOH.

3. The method of claim 2, wherein R² is a C₉ alkyl.

4. The method of claim 2, wherein R² is a branched alkyl.

5. The method of claim 2, wherein R² is 1,1-dimethylheptyl.

6. The method of claim 1, wherein the cannabinoid is ajulemic acid.

7. The method of claim 1, wherein the subject is sensitive to the cannabinoid.

8. The method of claim 1, wherein the disorder is an autoimmune disorder.

9. The method of claim 8, wherein the autoimmune disorder is diabetes mellitus, impaired glucose intolerance, diabetic retinopathy, obesity, systemic lupus erythematosis, rheumatoid arthritis, spondylo arthritis, asthma, inflammatory bowel disease, vasculitis, dermatomyositis, polymyositis, sjogren's syndrome, or hyperthyroidism.

10. The method of claim 1, wherein the disorder is ankylosing spondylitis, gout, arthritis associated with gout, osteoarthritis, osteoporosis, atherosclerosis, hypertension, hyperglycemia, coronary artery disease, or dyslipidemia.

11. The method of claim 1, wherein the disorder is exudative age-related macular degeneration or aereolar age-related macular degeneration.

12. The method of claim 1, wherein the disorder is an inflammatory disorder.

13. The method of claim 12, wherein the inflammatory disorder is rheumatoid arthritis, multiple sclerosis, myasthenia gravis, uveoretinitis, uveitis, iritis, cyclitis, choroiditis, chorioretinitis, vitritis, keratitis, conjunctivitis, psoriasis, eczema, thyroiditis, or a collagen vascular disorder.

14. The method of claim 13, wherein the collagen vascular disease is ankylosing spondylitis, lupus erythematosus, Reiter syndrome, Bechet disease, ulcerative colitis, Crohn's disease, or Wegener's granulomatosis.

15. The method of claim 1, wherein the mode of administration of the composition is oral, nasal, pulmonary, transdermal, or parenteral.

16. The method of claim 1, wherein the subject is a mammal.

17. The method of claim 16, wherein the mammal is a human, non-human primate, dog, cat, rodent, horse, cow, sheep, or goat.

18. A method of treating a subject having a disorder associated with peroxisome proliferator-activated receptor-gamma (PPARγ) function, the method comprising administering to the subject a pharmaceutically effective amount of a PPARγ activator.

19. The method of claim 18, wherein the PPARγ activator is a composition comprising a cannabinoid compound.

20. The method of claim 19, wherein the cannabinoid has the general formula: 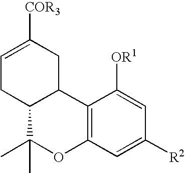
wherein R¹ is a hydrogen atom, —COCH₃ or —COCH₂CH₃, R² is a branched C₅-C₁₂ alkyl, and R³ is OH, OCH₃, or NHCH₂COOH.

21. The method of claim 19, wherein the cannabinoid is ajulemic acid.

22. A kit comprising a pharmaceutically effective amount of a composition comprising a cannabinoid compound and comprising instructions for use of the composition in treating a subject having a disorder associated with peroxisome proliferator-activated receptor-gamma (PPARγ) function.

23. The kit of claim 22, wherein the cannabinoid has the general formula: 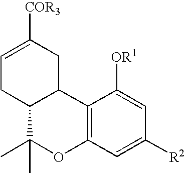
wherein R¹ is a hydrogen atom, —COCH₃ or —COCH₂CH₃, R² is a branched C₅-C₁₂ alkyl, and R³ is OH, OCH₃, or NHCH₂COOH.

24. The kit of claim 23, wherein R² is a C₉ alkyl.

25. The kit of claim 23, wherein R² is a branched alkyl.

26. The kit of claim 23, wherein R² is 1,1-dimethylheptyl.

27. The kit of claim 22 wherein the cannabinoid is ajulemic acid.

28. A composition for use as a medicament in treating a subject having a disorder associated with peroxisome proliferator-activated receptor-gamma (PPARγ) function, wherein the composition comprises a pharmaceutically effective amount of a cannabinoid compound.

29. Use of a composition for the manufacture of a medicament for use in treating a subject having a disorder associated with peroxisome proliferator-activated receptor-gamma (PPARγ) function, wherein the composition comprises a pharmaceutically effective amount of a cannabinoid compound.

30. A composition for use as a medicament in treating a subject having a disorder associated with peroxisome proliferator-activated receptor-gamma (PPARγ) function, wherein the composition comprises a pharmaceutically effective amount of a PPARγ activator.

31. Use of a composition for the manufacture of a medicament for use in treating a subject having a disorder associated with peroxisome proliferator-activated receptor-gamma (PPARγ) function, wherein the composition comprises a pharmaceutically effective amount of a PPARγ activator.
